(19) United States
(12) Patent Application Publication
(10) Pub. No.: US 2020/0123591 A1
(43) Pub. Date: Apr. 23, 2020

(54) EPIGENETIC PROFILING USING TARGETED CHROMATIN LIGATION

(71) Applicant: The Board of Trustees of the Leland Stanford Junior University, Stanford, CA (US)

(72) Inventors: Michael F. Clarke, Menlo Park, CA (US); Mark Zarnegar, Santa Clara, CA (US)

(21) Appl. No.: 16/628,992

(22) PCT Filed: Jul. 17, 2018
(86) PCT No.: PCT/US2018/042475

§ 371 Date: Jan. 6, 2020

Related U.S. Application Data
(60) Provisional application No. 62/533,502, filed on Jul. 17, 2017.

(51) Int. Cl. C12Q 1/6804; C12Q 1/6806; C12Q 1/6876; G01N 1/28; C12Q 1/6874

(52) U.S. Cl. CPC C12Q 1/6804; C12Q 1/6806; C12Q 1/6874; G01N 1/28; C12Q 1/6876

(57) ABSTRACT

Reagents and methods for epigenetic profiling using targeted chromatin ligation are disclosed. The method utilizes oligonucleotide adapters complexed with antibodies specific for DNA-binding proteins of interest and proximity ligation to tag fragmented chromatin with the adapters. Chromatin fragments having ligated adapters are amplified and sequenced with primers that hybridize to the adapters. This method can be used in epigenetic profiling, for example, for mapping histone modification patterns as well as transcriptional regulatory sites.

### STATEMENT REGARDING FEDERALLY SPONSORED RESEARCH OR DEVELOPMENT

This invention was made with Government support under contracts CA100225 and CA154209 awarded by the National Institutes of Health. The Government has certain rights in the invention.

### TECHNICAL FIELD

The present invention pertains generally to epigenetic profiling and chromatin mapping. In particular, the invention relates to reagents and methods for epigenetic profiling using targeted chromatin ligation with oligonucleotide adapters complexed with antibodies specific for DNA-binding proteins of interest.

### BACKGROUND

Chromatin immunoprecipitation (ChIP) combined with genome-wide next generation sequencing (ChIP-seq) has become an established research tool for investigating broad areas of biology. Standard ChIP-seq typically requires large numbers of cells (1-10 million), limiting its utility in situations when only small numbers of cells can be obtained. For example, biopsy specimens of human tissues are often limited to fewer than 50,000 cells. Moreover, organs and tissues contain complex mixtures of cells containing rare subpopulations, such as in bone marrow, where 1/20,000 cells are hematopoietic stem cells. Thus, applying ChIP-seq to understand biological processes such as stemness and differentiation has been hindered by the need for a large number of cells.

A number of techniques for applying ChIP-seq with low cell numbers (<100,000 cells) have been previously described, including methods optimized for fewer than 10,000 cells (Gilfillan et al. (2012) BMC Genomics 13:645, Adli et al. (2010) Nat. Methods 8:615-618, Shankaranarayanan et al. (2011) Nat. Methods 7:565-567, Schmidl et al. (2015) Nat. Methods 10:963-965, Zheng et al. (2015) Cell Rep 7:1505-1518, Lara-Astiaso et al. (2014) Immunogenetics Science 345:943-949, Brind'Amour et al. (2015) Nat. Commun. 6:6033, van Galen et al. (2016) Mol. Cell 1:170-80, and Cao et al. (2015) Nat. Methods 10:959-962). While some of these methods can increase the recovery of enriched material and improve the efficiency of immunoprecipitation for low cell counts (Zheng et al., supra; Cao et al., supra), they suffer from complicated or inefficient workflows that lead to loss of material at key steps (e.g., immunoprecipitation and washing). These losses, coupled with the small amounts of recovered material, further reduce ChIP-seq sensitivity (due in part to low efficiency conversion of enriched DNA to sequencing libraries). Moreover, methods for applying ChIP to less than 10,000 cells have been inconsistent or not demonstrated to work with some common histone marks ((Zheng et al., supra; Lara-Astiaso et al., supra; Brind'Amour et al., supra; van Galen et al., supra; Cao et al., supra). Attempts to overcome these shortcomings have produced prohibitively high methodological complexity, requiring an ever-increasing level of expertise for researchers to reproducibly execute protocols and obtain sufficient data quality with decreasing numbers of cells.

Thus, there remains a need for better methods of detecting epigenetic changes, particularly in rare cell populations.

### SUMMARY

The present invention relates to reagents and methods for epigenetic profiling using targeted chromatin ligation with oligonucleotide adapters complexed with antibodies specific for DNA-binding proteins of interest.

In one aspect, the invention includes a method of performing targeted chromatin ligation, the method comprising: a) providing a sample comprising chromatin; b) digesting the chromatin with one or more restriction enzymes, wherein cleavage of the chromatin occurs at positions that are not protected from the restriction enzymes by bound proteins; c) contacting the chromatin fragments with one or more antibody-adapter complexes, wherein each antibody-adapter complex comprises an antibody that specifically binds to a DNA-binding protein of interest complexed with an oligonucleotide adapter; d) ligating the oligonucleotide adapter of each antibody-adapter complex to its antibody-bound chromatin fragment; e) removing bound proteins from the chromatin fragments; f) amplifying ligated DNA from chromatin fragments having oligonucleotide adapters ligated at at least one end, wherein amplification is performed with at least one pair of primers that hybridize to the oligonucleotide adapters; and g) sequencing the amplified chromatin fragments.

In certain embodiments, the method further comprises isolating the chromatin fragments or diluting the chromatin fragments prior to amplifying the ligated DNA from the chromatin fragments having the oligonucleotide adapters ligated at at least one end.

In certain embodiments, digesting with restriction enzymes produces chromatin fragments ranging in size from about 250 base pairs to about 3000 base pairs. In certain embodiments, restriction enzymes are chosen that produce chromatin fragments having identical overhangs (e.g., dinucleotide, trinucleotide, or tetranucleotide overhangs), wherein the oligonucleotide adapter comprises an end sequence that is complementary to the identical overhangs of the chromatin fragments. In another embodiment, the restriction enzymes comprise one or more 4-base cutters, and/or 5-base cutters, and/or 6-base cutters. For example, the three 4-base cutters MseI, BfaI, and Csp6I can be used in combination with the 6-base cutter NdeI.

The chromatin can be from any type of eukaryotic cell, including a plant cell, an animal cell, a fungus cell, or a protist cell. The cell may be a live cell or a fixed cell. In one embodiment, the cell is a human cell.

The DNA-binding protein of interest may include, but is not limited to, a histone, transcription factor, or DNA modifying enzyme.

In certain embodiments, at least one antibody-adapter complex comprises an antibody selected from the group consisting of an anti-H3K4me3 antibody, an anti-H3K27me3 antibody, an anti-H3K36me3 antibody, and an anti-H3K27ac antibody.

In certain embodiments, at least some chromatin fragments are ligated to oligonucleotide adapters at both DNA ends.

In certain embodiments, chromatin fragments having oligonucleotide adapters ligated at one end are amplified. In other embodiments, chromatin fragments having oligonucleotide adapters ligated at both DNA ends are amplified. In another embodiment, ligated DNA is amplified from chromatin fragments having oligonucleotide adapters ligated at one end or at both ends.

Bound proteins can be removed from the chromatin fragments prior to sequencing, for example, by treating the chromatin fragments with a protease (e.g., proteinase K or trypsin) or protein denaturant.

In another embodiment, the method further comprises mapping sites of chromatin cleavage and locations of fragment sequences in the chromatin. Additionally, the method may further comprises producing a genome-wide profile of a DNA-binding protein of interest (e.g., genome-wide histone profile or transcription factor profile).

In certain embodiments, amplifying comprises performing polymerase chain reaction. The oligonucleotide adapters may comprise, for example, sequences for PCR primer binding sites, sequencing primer binding sites and/or indexing/barcoding sequences. In order to allow pooling of chromatin fragments from different cells or samples for high-throughput sequencing, chromatin fragments may be amplified with a set of primers comprising a barcode sequence to identify the eukaryotic cell or sample from which each amplified chromatin fragment originated.

In certain embodiments, the oligonucleotide adapters used in the antibody-adapter complexes are suitable for high-throughput sequencing. For example, the oligonucleotide adapters may be paired-end sequencing adapters or mate-pair sequencing adapters. In another embodiment, the method further comprises generating a paired-end or mate-pair sequencing library from the ligated chromatin fragments.

In antibody-adapter complexes, the antibody and the oligonucleotide adapter may be covalently or noncovalently connected. In certain embodiments, the oligonucleotide adapter further comprises a first member of a binding pair and the antibody further comprises a second member of a binding pair such that noncovalent binding between the first and second members of the binding pair joins the oligonucleotide adapter to the antibody in the antibody-adapter complex. For example, the binding pair may comprise streptavidin-biotin or avidin-biotin (e.g., biotinylated oligonucleotide adapter binds to streptavidin-antibody).

In another embodiment, the method further comprises genome-wide mapping of binding sites of DNA binding proteins, such as, but not limited to, transcription factors and associated proteins (e.g., that bind to promoters, enhancers, or silencers), histones, and enzymes (e.g., polymerases, DNA modifying enzymes).

In another embodiment, the method further comprises genome-wide profiling of histone modification patterns.

In another aspect, the invention includes a method of identifying an agent that modifies chromatin structure, the method comprising: a) treating a test sample comprising chromatin with the agent; b) providing a control sample comprising chromatin untreated with the agent; c) performing targeted chromatin ligation according to the methods described herein on the test sample and the control sample; and d) comparing the chromatin fragments from the test sample to the chromatin fragments from the control sample, wherein differences in the size or sequence of at least one chromatin fragment or a position of at least one cleavage site in the chromatin indicate that the agent has modified the structure of the chromatin. In certain embodiments, the method further comprises detecting differences in DNA histone modification or transcription factor binding in the test sample and the control sample.

These and other embodiments of the subject invention will readily occur to those of skill in the art in view of the disclosure herein.

### BRIEF DESCRIPTION OF THE FIGURES

FIGS. 1A-1C show the Targeted Chromatin Ligation (TCL) work flow and chromatin preparation. FIG. 1A shows the single tube TCL work flow (black box), which is followed by amplification and library construction. FIG. 1B shows gel analysis of restriction enzyme fragmented chromatin. A mix of four restriction enzymes was used to digest chromatin, as indicated. A representative gel of input from an MCF7 cell digest with conditions used for all TCL-seq replicates (see methods for details) and a representative gel of soluble MCF7 DNA (N-ChIP) input and the insoluble fraction are shown. FIG. 1C shows that the TCL-qPCR signal to noise ratio is not sensitive to reaction parameters when qPCR analysing all ligated chromatin fragments after T7 based amplification. TCL-qPCR data was first normalized to input, then normalized against a negative region to generate normalized fold signal. Gray bars represent signal from TCL reactions using three different molecular ratios of biotinylated adapter bound to streptavidin conjugated antibody (2:1, 3:1, and 4:1, respectively). Black bars represent signal from TCL reactions using three different quantities of antibody loaded with adapter at a 2:1 ratio (200 ng, 400 ng, and 600 ng, respectively). Data shown is the mean of three or more TCL replicates. Error bars represent S.D. Each bar represents a different genomic region predicted to be negative or positive for H3K27me3 modifications using ENCODE data tracks, and two primer sets for non-overlapping regions of the same gene were used to gauge consistent coverage.

FIGS. 2A-2E show analysis of a critical TCL reaction parameter. FIG. 2A shows a schematic and TCL-qPCR data from optimization of a critical parameter of TCL reactions is provided. The TCL-qPCR data is shown as fold signal described above. Black and gray data bars were generated with different adapter to antibody molecular ratios (2:1 and 1:1, respectively). Data shown as mean plus S.D. from three independent TCL replicates. FIG. 2B shows qPCR data (mean from technical replicates, no error bars provided) from a single representative 2000 cell TCL reaction for H3K27me3 is shown. Data presented as described above. FIG. 2C shows qPCR data (mean from technical replicates, no error bars provided) from a representative one million cell N-ChIP sample for H3K27me3 is shown. Data presented as described above. FIG. 2D shows gel analysis of amplification products from TCL reactions performed using input, no antibody, or antibodies as labelled. FIG. 2E shows a representative amplification product further digested by restriction enzymes and used to produce a sequencing library.

FIGS. 3A-3D show that TCL-seq generates high quality data for multiple histone marks and only 200 cells. FIG. 3A shows normalized 2000 cell TCL-seq data for H3K27me3 across a random genomic window is shown in comparison to ChIP-seq data. N-ChIP-seq data was generated using a million cells or 2000 cells. ENCODE data was generated using ˜1 million cells. FIG. 3B shows normalized 2000 cell TCL-seq data for H3K36me3 is presented as described above. FIG. 3C shows normalized TCL-seq data for H3K27ac, which is shown in comparison to ChIP-seq data as described above. TCL-seq and N-ChIP-seq samples were generated with decreasing cell numbers (10,000, 2,000, 400 and 200 cells. FIG. 3D shows TCL-seq data produced with less than 1,000 neurosphere cells (˜1,000 sorted events) are shown for three different histone marks. H3K36me3 and H3K27me3 signal tracks were generated using all uniquely mapped reads. H3K4me3 signal tracks show only reads across promoters (5,000 bp around TSSs).

FIGS. 4A-4C show correlation and principal component analysis (PCA) of genome-wide data. FIG. 4A shows a heat map with Pearson correlations for MCF7 data comparing ENCODE ChIP, N-ChIP, and TCL. FIG. 4B shows a heat map showing Pearson correlations for neurosphere TCL data. FIG. 4C shows principal component analysis of 2000 cell TCL data and ENCODE ChIP data. PCA data was generated using Deeptools and a 500 bp bin size. Heat maps of Pearson correlations for TCLs (2000 cell samples), N-ChIP (2000 cell and 1,000,000 cell samples), and ENCODE ChIP data were generated with Deeptools in Galaxy using 2 kb bins.

FIGS. 5A-5D show the advantages of the single adapter TCL reaction. FIG. 5A shows a schematic depicting ligation products of TCL reactions using the dual adapter strategy. FIG. 5B shows a schematic depicting the ligation products of single adapter TCL reactions. FIG. 5C shows a representative image of gel analyzed DNA amplified from dual adapter TCL reactions. The arrow indicates primer dimer PCR artefacts. FIG. 5D shows a representative image of gel analyzed DNA amplified from single adapter TCL reactions. The arrow indicates the absence of primer dimer PCR artefacts. The DNA appears smeared due to a large portion of the product being single stranded due to head to tail annealing preventing formation of double stranded DNA during amplification.

FIGS. 6A-6C show a TCL plate-based protocol, which can be performed without a column purification step. FIG. 6A shows a schematic of the TCL workflow. FIGS. 6B and 6C show qPCR data (mean from technical replicates, no error bars provided) from a single representative TCL reaction for H3K27me3 performed with the plate-based protocol using 200 cells (FIG. 6B) or 25 cells (FIG. 6C) as indicated.

### DETAILED DESCRIPTION OF THE INVENTION

The practice of the present invention will employ, unless otherwise indicated, conventional methods of molecular biology, chemistry, and biochemistry within the skill of the art. Such techniques are explained fully in the literature. See, e.g., Chromatin Protocols (Methods in Molecular Biology, S. P. Chellappan ed., Humana Press, 2^{nd} edition, 2009); Nucleosomes, Histones & Chromatin Part B, Volume 513 (Methods in Enzymology, Academic Press, 2012); B. M. Turner Chromatin and Gene Regulation: Molecular Mechanisms in Epigenetics (Wiley-Blackwell, 2002); C. Carlberg and F. Molnar Mechanisms of Gene Regulation (Springer, 2^{nd} edition, 2016); Next Generation Sequencing: Translation to Clinical Diagnostics (L. C. Wong ed., Springer, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### 1. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a nucleic acid” includes a mixture of two or more such nucleic acids, and the like.

The term “about,” particularly in reference to a given quantity, is meant to encompass deviations of plus or minus five percent.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” to an antibody or “specifically (or selectively) immunoreactive with,” when referring to an antigen (e.g., DNA-binding protein), refers to a binding reaction that is determinative of the presence of the antigen in a heterogeneous population of proteins and other biologics. Thus, under designated immunoassay conditions, the specified antibodies bind to a particular antigen at least two times the background and do not substantially bind in a significant amount to other antigens present in the sample. Specific binding to an antibody under such conditions may require an antibody that is selected for its specificity for a particular antigen. For example, polyclonal antibodies raised to an antigen from specific species such as rat, mouse, or human can be selected to obtain only those polyclonal antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory

Manual (1988), for a description of immunoassay formats and conditions that can be used to determine specific immunoreactivity). Typically, a specific or selective reaction will be at least twice background signal or noise and more typically more than 10 to 100 times background.

“Substantially purified” generally refers to isolation of a substance (compound, polynucleotide, oligonucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides oligonucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein, the term “biological sample” includes any cell or tissue or bodily fluid containing nucleic acids from a eukaryotic or prokaryotic organism, such as cells from plants, animals, fungi, protists, bacteria, or archaea. The biological sample may include cells from a tissue or bodily fluid, including but not limited to, blood, saliva, cells from buccal swabbing, fecal matter, urine, bone marrow, spinal fluid, lymph fluid, skin, organs, and biopsies, as well as in vitro cell culture constituents, including recombinant cells and tissues grown in culture medium. A biological sample may also include a viral particle comprising nucleic acids.

The term “barcode” refers to a nucleic acid sequence that is used to identify a single cell, a subpopulation of cells, or a sample. Barcode sequences can be linked to a target nucleic acid of interest during amplification and used to trace back the amplicon to the cell or sample from which the target nucleic acid originated. A barcode sequence can be added to a target nucleic acid of interest during amplification by carrying out PCR with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid.

As used herein, the term “binding pair” refers to first and second molecules that specifically bind to each other. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the development of a novel method for epigenetic profiling using targeted chromatin ligation. The method utilizes oligonucleotide adapters complexed with antibodies specific for DNA-binding proteins of interest (e.g., histones, transcription factors, or DNA-modifying enzymes) and proximity ligation to tag fragmented chromatin with the adapters. Chromatin fragments having ligated adapters can be amplified and sequenced with primers that hybridize to the adapters. This method can be used, for example, for mapping histone modification patterns as well as regulatory binding sites of transcription factors and associated proteins (e.g., promoters, enhancers, or silencers).

In order to further an understanding of the invention, a more detailed discussion is provided below regarding targeted chromatin ligation and its use in epigenetic profiling.

A. Targeted Chromatin Ligation

In one aspect, the invention includes a method of performing targeted chromatin ligation. The method generally comprises: a) providing a sample comprising chromatin; b) digesting the chromatin with one or more restriction enzymes, wherein cleavage of the chromatin occurs at positions that are not protected from the restriction enzymes by bound proteins; c) contacting the chromatin fragments with one or more antibody-adapter complexes, wherein each antibody-adapter complex comprises an antibody that specifically binds to a DNA-binding protein of interest complexed with an oligonucleotide adapter; d) ligating the oligonucleotide adapter of each antibody-adapter complex to its bound chromatin fragment; e) removing bound proteins from the chromatin fragments; f) isolating the chromatin fragments; g) amplifying the chromatin fragments comprising ligated oligonucleotide adapters, wherein amplification is performed with at least one pair of primers that hybridize to the oligonucleotide adapters; and h) sequencing the amplified chromatin fragments.

Targeted chromatin ligation can be used to analyze chromatin from any eukaryotic organism, including plants, animals, fungi, and protists. The chromatin can be from a biological sample containing cells, tissue, or a bodily fluid, including but not limited to, blood, saliva, cells from buccal swabbing, fecal matter, urine, bone marrow, spinal fluid, lymph fluid, skin, organs, and biopsies, or in vitro cell culture constituents, including recombinant cells and tissues grown in culture medium. In certain embodiments, the chromatin is from a cell, such as an invertebrate cell, vertebrate cell, plant cell, yeast cell, mammalian cell, rodent cell, primate cell, or human cell. The methods can be applied to living cells or fixed cells.

Cells may be pre-treated in any number of ways prior to performing targeted chromatin ligation. For instance, in certain embodiments, the cell may be treated to disrupt (or lyse) the cell membrane, for example, by treating samples with one or more detergents (e.g., Triton-X-100, sodium deoxycholate, sarkosyl, Tween 20, Igepal CA-630, NP-40, Brij 35, and sodium dodecyl sulfate). In cell types with cell walls, such as yeast and plants, initial removal of the cell wall may be necessary to facilitate cell lysis. Cell walls can be removed, for example, using enzymes, such as cellulases, chitinases, or bacteriolytic enzymes, such as lysozyme (destroys peptidoglycans), mannase, and glycanase. As will be clear to one of skill in the art, the selection of a particular enzyme for cell wall removal will depend on the cell type under study.

Additionally, cells may be fixed prior to performing targeted chromatin ligation. For instance, in certain embodiments, cells may be fixed with one or more crosslinking agents such as formaldehyde, gluteraldehyde, or bifunctional linkers such as ethylene glycol bis(succinimidyl succinate (EGS); or fixed by dehydration with alcohols such as methanol or ethanol.

Antibodies specific for DNA binding proteins of interest may be any type of antibody, including polyclonal and monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)2 and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule (i.e., specifically binds to a target DNA-binding protein of interest).

Antibody-adapter complexes of the subject methods include an oligonucleotide molecule and an antibody specific for a DNA-binding protein of interest. The oligonucleotide adapter may vary depending, in part, on the amplification and/or sequencing method employed, the method of complexation with the antibody, the specific DNA-binding protein to be detected, etc. The oligonucleotide adapter can be designed to have an overhang sequence compatible with ligation to the DNA ends of chromatin fragments produced from enzymatic digestion by restriction enzymes as discussed further below.

Generally, the length of the oligonucleotide adapter will be at least 15 nucleotides, but may range from 15 nucleotides to 200 nucleotides or more including but not limited to e.g., 20 or more nucleotides, 25 or more nucleotides, 30 or more nucleotides, 35 or more nucleotides, 40 or more nucleotides, 45 or more nucleotides, 50 or more nucleotides, 55 or more nucleotides, 60 or more nucleotides, 65 or more nucleotides, 70 or more nucleotides, 75 or more nucleotides, 80 or more nucleotides, 90 or more nucleotides, 95 or more nucleotides, 100 or more nucleotides, 15 to 200 nucleotides, 20 to 200 nucleotides, 25 to 200 nucleotides, 30 to 200 nucleotides, 35 to 200 nucleotides, 40 to 200 nucleotides, 45 to 200 nucleotides, 50 to 200 nucleotides, 15 to 100 nucleotides, 20 to 100 nucleotides, 25 to 100 nucleotides, 30 to 100 nucleotides, 35 to 100 nucleotides, 40 to 100 nucleotides, 45 to 100 nucleotides, 50 to 100 nucleotides, 55 to 100 nucleotides, 60 to 100 nucleotides, etc.

In some instances, an oligonucleotide adapter of the subject disclosure may include one or more nucleoside analogs. For example, in some instances, an oligonucleotide adapter may include one or more deoxyribouracil (i.e., deoxyribose uracil,—deoxyuridine, etc.) nucleosides/nucleotides. In certain instances, a bridging polynucleotide may include 2 or more nucleoside analogs including but not limited to e.g., 3 or more, 4 or more, 5 or more, 6 or more, etc. In some instances, the number of nucleoside analogs as a percentage of the total bases of the oligonucleotide is 1% or more, including but not limited to e.g., 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, etc.

The oligonucleotide adapter may be complexed with or conjugated to an antibody by any convenient method. In certain embodiments, the oligonucleotide adapter is conjugated to a first member of a binding pair and the antibody is conjugated to a second member of a binding pair, wherein noncovalent binding between the first and second members of the binding pair joins the oligonucleotide adapter to the antibody in an antibody-adapter complex. Exemplary binding pairs include biotin-avidin, biotin-streptavidin, hormone-receptor, receptor-receptor agonist or antagonist, lectin-carbohydrate, enzyme-enzyme cofactor, enzyme-enzyme-inhibitor, hapten-antibody, and complementary polynucleotide pairs capable of forming nucleic acid duplexes, and the like. The oligonucleotide adapter or antibody may be linked directly to the specific-binding molecule or linked indirectly through a chemical linker. In one embodiment, the binding pair comprises streptavidin-biotin or avidin-biotin (e.g., biotinylated oligonucleotide adapter binds to streptavidin-antibody to form complex).

In certain embodiments, the oligonucleotide adapters are designed to facilitate high-throughput amplification and/or sequencing. For example, adapters may comprise a common priming site to allow massively parallel sequencing. Adapters may further comprise common 5′ and 3′ priming sites to allow amplification of DNA fragments in parallel with a set of universal primers. In addition, adapters may comprise indexing/barcoding sequences to identify the cell or sample from which each chromatin fragment originated to allow pooling of DNA fragments from different cells or samples for high-throughput amplification and/or sequencing. In one embodiment, the adapters are paired-end sequencing adapters (illumina.com/technology/technology/next-generation-sequencing/paired-end-sequencing_assay.html). In another embodiment, long chromatin fragments that are incompatible with some sequencing platforms are circularized by ligation to adapters for mate-pair sequencing (see, e.g., illumina.com/technology/next-generation-sequencing/mate-pair-sequencing_assay.html).

Chromatin fragments are produced by digesting the DNA with one or more restriction enzymes, which catalyze hydrolysis of phosphodiester bonds in the chromatin DNA to produce double stranded breaks. In particular, type II restriction enzymes can be used that selectively recognize short, usually palindromic nucleotide sequences (e.g., 4 to 8 nucleotides). Restriction enzymes are selected to provide DNA ends to the chromatin fragments compatible with ligation of an oligonucleotide adapter. For example, restriction enzymes can be chosen that produce a staggered cut (“sticky end”) such that the generated fragments have single-stranded tails (i.e., overhangs) comprising sequences that are complementary and capable of hybridizing with an overhang sequence of an oligonucleotide adapter. In certain embodiments, one or more restriction enzymes are used that produce chromatin fragments having identical overhangs (e.g., dinucleotide, trinucleotide, or tetranucleotide overhangs). Alternatively, restriction enzymes can be chosen that produce a blunt end by cutting both strands of the DNA at the same position. In order to improve ligation efficiency, blunt ends can be modified using a terminal deoxynuclotidyl transferase to add nucleotides to the DNA ends to provide a sequence complementary to an overhang sequence of an oligonucleotide adapter. The selection of restriction enzymes is a matter of choice, but preferably, digestion is performed under conditions that produce chromatin fragments ranging in size from about 250 base pairs to about 3000 base pairs.

In certain embodiments, the restriction enzymes comprise one or more 4-base cutters, and/or 5-base cutters, and/or 6-base cutters. Restriction enzymes that recognize 6 bp sequences of DNA (i.e., 6-base cutters) include, but are not limited to, AclI, HindIII, SspI, BspLU11I, AgeI, MluI, SpeI, BglII, Eco47III, StuI, ScaI, ClaI, AvaIII, VspI, MfeI, PmaCI, PvuII, NdeI, NcoI, SmaI, SadI, AvrII, PvuI, XmaIII, SplI, XhoI, PstI, AflII, EcoRI, AatII, Sad, EcoRV, SphI, Nad, BsePI, NheI, BamHI, NarI, ApaI, KpnI, SnaI, SalI, ApaLI, HpaI, SnaBI, BspHI, BspMII, NruI, XbaI, BclI, MstI, BalI, Bsp1407I, PsiI, AsuII and AhaIII. Restriction enzymes that recognize 4 or 5 bp sequences of DNA (i.e., 4-base cutters and 5-base cutters) include, but are not limited to, MseI, BfaI, Csp6I, TspEI, MaeII, AluI, NlaIII, HpaII, FnuDII, MaeI, DpnI, MboI, HhaI, HaeIII, RsaI, TaqI, CviRI, Sth132I, AciI, DpnII, Sau3AI and MnII.

In certain embodiments, one, two, three, four, or five 4-base cutters and one, two, three, four, or five 6-base cutters are used. In another embodiment, the three 4-base cutters, MseI, BfaI, and Csp6I are used in combination with the 6-base cutter, NdeI.

Binding of the antibody-adapter complexes to target DNA-binding proteins of interest is followed by ligation of the conjugated oligonucleotide adapter to its antibody-bound chromatin fragment. A DNA ligase is used to join the conjugated oligonucleotide adapter to the chromatin DNA at the fragment ends. DNA ligase acts by catalyzing the formation of a phosphodiester bond between the oligonucleotide adapter and the DNA of a chromatin fragment. Ligation occurs when an oligonucleotide adapter is located in proximity to a DNA end of a chromatin fragment as a result of binding by a chosen antibody specific for a DNA-binding protein of interest. Oligonucleotide adapters may be ligated to the 5′-end, the 3′-end, or at both ends (i.e., doubly ligated) of a chromatin fragment using a DNA ligase (e.g., T4 DNA ligase). Ligated DNA can be enriched through selective amplification using primers that hybridize to the oligonucleotide adapters. In some embodiments, only doubly ligated chromatin fragments are amplified using universal primers that hybridize to the oligonucleotide adapters.

The ligated DNA can be amplified prior to sequencing using any method for amplifying nucleic acids, including, but not limited to polymerase chain reaction (PCR), isothermal amplification, ligase chain reaction (LCR), strand displacement amplification (SDA), and helicase-dependent amplification (HDA).

In some instances, amplification is performed by polymerase chain reaction (PCR), which is a technique for amplifying a desired target nucleic acid sequence contained in a nucleic acid molecule or mixture of molecules. A pair of primers is employed in excess, which hybridize to the complementary strands of the target nucleic acid (e.g., chromatin fragment DNA at the site of a ligated adapter). The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

Any oligonucleotide primers with which the template nucleic acid (hereinafter referred to as template DNA for convenience) is contacted will be of sufficient length to provide for hybridization to complementary template DNA under annealing conditions. The primers will generally be at least 6 bp in length, including but not limited to e.g., at least 10 bp in length, at least 15 bp in length, at least 16 bp in length, at least 17 bp in length, at least 18 bp in length, at least 19 bp in length, at least 20 bp in length, at least 21 bp in length, at least 22 bp in length, at least 23 bp in length, at least 24 bp in length, at least 25 bp in length, at least 26 bp in length, at least 27 bp in length, at least 28 bp in length, at least 29 bp in length, at least 30 bp in length, and may be as long as 60 bp in length or longer, where the length of the primers will generally range from 18 to 50 bp in length, including but not limited to, e.g., from about 20 to 35 bp in length. In some instances, the template DNA may be contacted with a single primer or a set of two primers (forward and reverse primers), depending on whether primer extension, linear or exponential amplification of the template DNA is desired. Methods of PCR that may be employed in the subject methods include but are not limited to those described in U.S. Pat. Nos. 4,683,202; 4,683,195; 4,800,159; 4,965,188 and 5,512,462, the disclosures of which are herein incorporated by reference.

In addition to the above components, a PCR reaction mixture produced in the subject methods may include a polymerase and deoxyribonucleoside triphosphates (dNTPs). The desired polymerase activity may be provided by one or more distinct polymerase enzymes. In many embodiments, the reaction mixture includes at least a Family A polymerase, where representative Family A polymerases of interest include, but are not limited to: Thermus aquaticus polymerases, including the naturally occurring polymerase (Taq) and derivatives and homologues thereof, such as Klentaq (as described in Proc. Natl. Acad. Sci USA (1994) 91:2216-2220, the disclosure of which is incorporated herein by reference in its entirety); Thermus thermophilics polymerases, including the naturally occurring polymerase (Tth) and derivatives and homologues thereof, and the like. In certain embodiments where the amplification reaction that is carried out is a high fidelity reaction, the reaction mixture may further include a polymerase enzyme having 3′-5′ exonuclease activity, e.g., as may be provided by a Family B polymerase, where Family B polymerases of interest include, but are not limited to: Thermococcus litoralis DNA polymerase (Vent) (e.g., as described in Perler et al., Proc. Natl. Acad. Sci. USA (1992) 89:5577, the disclosure of which is incorporated herein by reference in its entirety); Pyrococcus species GB-D (Deep Vent); Pyrococcus furiosus DNA polymerase (Pfu) (e.g., as described in Lundberg et al., Gene (1991) 108: 1-6, the disclosure of which is incorporated herein by reference in its entirety), Pyrococcus woesei (Pwo) and the like. Generally, the reaction mixture will include four different types of dNTPs corresponding to the four naturally occurring bases are present, i.e. dATP, dTTP, dCTP and dGTP and in some instances, may include one or more modified nucleotide dNTPs.

A PCR reaction will generally be carried out by cycling the reaction mixture between appropriate temperatures for annealing, elongation/extension, and denaturation for specific times. Such temperature and times will vary and will depend on the particular components of the reaction including, e.g., the polymerase and the primers as well as the expected length of the resulting PCR product. In some instances, e.g., where nested or two-step PCR are employed the cycling-reaction may be carried out in stages, e.g., cycling according to a first stage having a particular cycling program or using particular temperature(s) and subsequently cycling according to a second stage having a particular cycling program or using particular temperature(s).

Multistep PCR processes may or may not include the addition of one or more reagents following the initiation of amplification. For example, in some instances, amplification may be initiated by elongation with the use of a polymerase and, following an initial phase of the reaction, additional reagent(s) (e.g., one or more additional primers, additional enzymes, etc.) may be added to the reaction to facilitate a second phase of the reaction. In some instances, amplification may be initiated with a first primer or a first set of primers and, following an initial phase of the reaction, additional reagent(s) (e.g., one or more additional primers, additional enzymes, etc.) may be added to the reaction to facilitate a second phase of the reaction. In certain embodiments, the initial phase of amplification may be referred to as “preamplification”.

In some instances, amplification may be carried out under isothermal conditions, e.g., by means of isothermal amplification. Methods of isothermal amplification generally make use of enzymatic means of separating DNA strands to facilitate amplification at constant temperature, such as, e.g., strand-displacing polymerase or a helicase, thus negating the need for thermocycling to denature DNA. Any convenient and appropriate means of isothermal amplification may be employed in the subject methods including but are not limited to: loop-mediated isothermal amplification (LAMP), strand displacement amplification (SDA), helicase-dependent amplification (HDA), nicking enzyme amplification reaction (NEAR), and the like. LAMP generally utilizes a plurality of primers, e.g., 4-6 primers, which may recognize a plurality of distinct regions, e.g., 6-8 distinct regions, of target DNA. Synthesis is generally initiated by a strand-displacing DNA polymerase with two of the primers forming loop structures to facilitate subsequent rounds of amplification. LAMP is rapid and sensitive. In addition, the magnesium pyrophosphate produced during the LAMP amplification reaction may, in some instances be visualized without the use of specialized equipment, e.g., by eye. SDA generally involves the use of a strand-displacing DNA polymerase (e.g., Bst DNA polymerase, Large (Klenow) Fragment polymerase, Klenow Fragment (3′-5′ exo-), and the like) to initiate at nicks created by a strand-limited restriction endonuclease or nicking enzyme at a site contained in a primer. In SDA, the nicking site is generally regenerated with each polymerase displacement step, resulting in exponential amplification. HDA generally employs: a helicase which unwinds double-stranded DNA unwinding to separate strands; primers, e.g., two primers, that may anneal to the unwound DNA; and a strand-displacing DNA polymerase for extension. NEAR generally involves a strand-displacing DNA polymerase that initiates elongation at nicks, e.g., created by a nicking enzyme. NEAR is rapid and sensitive, quickly producing many short nucleic acids from a target sequence.

In some instances, entire amplification methods may be combined or aspects of various amplification methods may be recombined to generate a hybrid amplification method. For example, in some instances, aspects of PCR may be used, e.g., to generate the initial template or amplicon or first round or rounds of amplification, and an isothermal amplification method may be subsequently employed for further amplification. In some instances, an isothermal amplification method or aspects of an isothermal amplification method may be employed, followed by PCR for further amplification of the product of the isothermal amplification reaction. In some instances, a sample may be preamplified using a first method of amplification and may be further processed, including e.g., further amplified or analyzed, using a second method of amplification. As a non-limiting example, a sample may be preamplified by PCR and further analyzed by qPCR.

In some instances, the amplification step and the detection step, described below, may be combined, with or without the use of a preamplifcation step. In some instances, the particular amplification method employed allows for the qualitative detection of amplification product, e.g., by visual inspection of the amplification reaction with or without a detection reagent. In one embodiment, the ligation products are amplified by isothermal amplification, e.g., LAMP, and the amplification generates a visual change in the amplification reaction indicative of efficient amplification and thus presence of the antibody isotype in the sample. In some instances, the amplification and detection steps are combined by monitoring the amplification reaction during amplification such as is performed in, e.g., real-time PCR, also referred to herein as quantitative PCR (qPCR).

The methods of the invention can be adapted to multiplexing. For example, a plurality of antibody-adapter complexes can be added to a sample, wherein the adapters are complexed with antibodies specific for different DNA-binding proteins. Each adapter may comprise a barcode identifying the particular DNA-binding protein the conjugated antibody is targeting. In certain embodiments, targeted chromatin ligation is performed with one or more antibody-adapter complexes comprising antibodies specific for one or more histone proteins or histone modifications (e.g., methylation (me), acetylation (ac), or phosphorylation). For example, targeted chromatin ligation may be performed with a set of antibody-adapter complexes comprising one or more antibodies selected from the group consisting of an anti-H3K4me3 antibody, an anti-H3K27me3 antibody, an anti-H3K36me3 antibody, and an anti-H3K27ac antibody. The oligonucleotides in the conjugates may comprise antibody-specific DNA barcodes that can be amplified and detected simultaneously by using a suitable combination of primers and/or probes in a multiplex-type assay format.

Exemplary DNA sequences for oligonucleotide adapters and PCR primers for detection of the chromatin ligation products are shown in Example 1 and Tables 2 and 3.

Primers and oligonucleotide adapters can be readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

Additionally, barcode sequences can be added to amplicon products to identify the cell or sample from which each amplified chromatin fragment originated. The use of barcodes allows chromatin fragments from different cells or samples to be pooled in a single reaction mixture for sequencing while still being able to trace back a particular chromatin fragment to the particular cell or sample from which it originated. Each cell or sample is identified by a unique barcode sequence comprising at least five nucleotides. A barcode sequence can be added during amplification by carrying out PCR with a primer that contains a region comprising the barcode sequence and a region that is complementary to the ligated oligonucleotide adapter such that the barcode sequence is incorporated into the final amplified product. Barcode sequences can be added at one or both ends of an amplicon.

Moreover, oligonucleotides, particularly primers or probes may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al., Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al., Nucl. Acids Res. (1985) 13:4485-4502 and Spoat et al. Nucl. Acids Res. (1987) 15:4837-4848. A comprehensive review of methodologies for labeling DNA fragments is provided in Matthews et al., Anal. Biochem. (1988) 169:1-25.

For example, oligonucleotides may be fluorescently labeled by linking a fluorescent molecule to the non-ligating terminus of the molecule. Guidance for selecting appropriate fluorescent labels can be found in Smith et al., Meth. Enzymol. (1987) 155:260-301; Karger et al., Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11^{th} edition, Johnson and Spence eds., 2010 (Molecular Probes/Life Technologies). Fluorescent labels include fluorescein and derivatives thereof, such as disclosed in U.S. Pat. No. 4,318,846 and Lee et al., Cytometry (1989) 10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No.4,174,384. Additional dyes include SYBR green, SYBR gold, Yakima Yellow, Texas Red, 3-(ε-carboxypentyl)-3′-ethyl-5,5′-dimethyloxa-carbocyanine (CYA); 6-carboxy fluorescein (FAM); CAL Fluor Orange 560, CAL Fluor Red 610, Quasar Blue 670; 5,6-carboxyrhodamine-110 (R110); 6-carboxyrhodamine-6G (R6G); N′,N′,N′,N′-tetramethyl-6-carboxyrhodamine (TAMRA); 6-carboxy-X-rhodamine (ROX); 2′, 4′, 5′, 7′, -tetrachloro-4-7-dichlorofluorescein (TET); 2′, 7′-dimethoxy-4′, 5′-6 carboxyrhodamine (JOE); 6-carboxy-2′,4,4′,5′,7,7′-hexachlorofluorescein (HEX); Dragonfly orange; ATTO-Tec; Bodipy; ALEXA; VIC, Cy3, and Cy5. These dyes are commercially available from various suppliers such as Life Technologies (Carlsbad, Calif.), Biosearch Technologies (Novato, Calif.), and Integrated DNA Technolgies (Coralville, Iowa). Fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Oligonucleotides can also be labeled with a minor groove binding (MGB) molecule, such as disclosed in U.S. Pat. Nos. 6,884,584, 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al., (1995) “Detection of Acridinium Esters by Chemiluminescence” in Nonisotopic Probing, Blotting and Sequencing, Kricka L. J.(ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

After ligation to adapters, bound proteins can be removed from the chromatin fragments prior to sequencing, for example, by treating the chromatin fragments with a broad-spectrum protease (e.g., proteinase K) or protein denaturant.

B. Sequencing of Nucleic Acids

Any high-throughput technique for sequencing can be used in the practice of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence chromatin fragments.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina MiSeq, NextSeq, and HiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

Long-read sequencing methods are also of interest, which may be used for sequencing fragments longer than 1 kilobase. Such long-read methods may include Oxford Nanopore MinION sequencing, Pacific Biosciences RS single molecule, real-time (SMRT) sequencing, and Illumina synthetic long-read sequencing (see, e.g., Koren et al. (2015) Curr. Opin. Microbiol. 23:110-120, Lu et al. (2016) Genomics Proteomics Bioinformatics 14(5):265-279, Eid et al. (2009) Science 323:133-138, Chin et al. (2011) N. Engl. J. Med. 364:33-42, Voskoboynik et al. (2013) Elife 2:e00569; herein incorporated by reference).

In addition, mate-pair sequencing can be used for sequencing longer fragments. In mate-pair sequencing, long fragments are circularized by ligation to the ends of a common DNA adaptor having a known sequence, and a sequencing library of fragments is generated that retains the adaptor mated to only the ends of the original fragments (see, e.g., Mardis et al. (2016) Cold Spring Harb Protoc. 2016 Nov. 1 [Epub ahead of print], Gao et al. (2015) Viruses 7(8):4507-4528, Yang et al. (2014) Cancer Inform. 13(Suppl 2):49-53; herein incorporated by reference)

C. Applications

The methods of the present invention make possible detailed study of the relationship between chromatin structure and regulation of gene expression and will find numerous applications in basic research and development. In particular, the technology allows genome-wide mapping of chromatin structure, including mapping positions of nucleosomes and histone modifications, and detection of binding sites of DNA binding proteins, such as, but not limited to, transcription factors and associated proteins (e.g., that bind to promoters, enhancers, or silencers), histones, and enzymes (e.g., polymerases, DNA modifying enzymes).

The methods of the invention can be used, for example, for detecting genome wide histone modifications. For example, antibody-adapter complexes comprising antibodies specific for particular histone modifications (e.g., an anti-H3K4me3 antibody, an anti-H3K27me3 antibody, an anti-H3K36me3 antibody, or an anti-H3K27ac) antibody) can be used. Multiple barcoded adapter-antibody complexes can be used for multiplex detection of more than one histone modification in the same chromatin sample.

The methods of the invention can also be used for mapping the positions of nucleosomes, transcription factors, and other DNA binding proteins as well as studying the dynamics of nucleosome repositioning during chromatin remodeling in vivo. Chromatin accessibility controls binding of transcription factors to DNA and, in turn, gene expression. In regions of open chromatin having increased accessibility, transcription factors compete with nucleosomes for binding to regulatory regions of the DNA. Thus, the methods of the invention are useful for identifying epigenetic changes associated with altered gene regulation.

The methods of the invention will be especially useful for detecting chromatin structural changes associated with physiological processes and diseases. For example, chromatin from certain diseased cells or tissues may have an altered chromatin structure relative to the chromatin from normal or healthy cells. Therefore, analysis of chromatin structure from such diseased cells or tissues may be useful for diagnosing a disease. Epigenetic changes may also be associated with disease progression; therefore, detecting such changes may be useful for monitoring disease progression. Furthermore, detecting epigenetic changes associated with a disease may be useful for identifying potential therapeutic targets for treatment.

The methods of the invention can also be used for identifying agents capable of modifying chromatin structure. For example, a test sample comprising chromatin treated with an agent and a comparable control sample comprising chromatin untreated with the agent can be analyzed using targeted chromatin ligation as described herein. The resulting adapter-ligated DNA fragments from the test sample can be compared to the adapter-ligated DNA fragments from the control sample, wherein differences in the size or sequence of at least one DNA fragment or a position of at least one cleavage site in the chromatin indicate that the agent has modified the structure of the chromatin in the test sample.

In addition, the methods of the invention can be combined with any other method for analyzing chromatin structure, including, but not limited to, DNase I hypersensitive sites sequencing (DNase-seq), which uses DNase Ito map regions in the genome accessible to DNase I, formaldehyde-assisted isolation of regulatory elements sequencing (FAIRE-seq), which identifies nucleosome depleted regions in the genome, and assay for transposable accessible chromatin sequencing (ATAC-seq), which uses the Tn5 transposase to insert transposons into accessible regions of the genome.

D. Kits

The above-described compositions, including the oligonucleotide adapters, antibodies specific for DNA-binding proteins of interest, primers, restriction enzymes, and optionally other reagents for performing nucleic acid amplification, such as by PCR or isothermal amplification, and sequencing, can be provided in kits, with suitable instructions and other necessary reagents, in order to perform targeted chromatin ligation, as described above. The kit will normally contain in separate containers the oligonucleotide adapters, antibodies specific for DNA-binding proteins of interest, primers, restriction enzymes, and other reagents that the method requires. Instructions (e.g., written, CD-ROM, DVD, Blu-ray, flash drive, hyperlink for digital download, etc.) for carrying out targeted chromatin ligation usually will be included in the kit. The kit can also contain other packaged reagents and materials (e.g., buffers, nucleotides, polymerases, and the like).

In certain embodiments, the kit comprises at least one adapter-antibody complex comprising a biotinylated oligonucleotide adapter and a streptavidin-conjugated antibody. In another embodiment, the kit comprises restriction enzymes, including the three 4-base cutters, MseI, BfaI, and Csp6I and the 6-base cutter, NdeI.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Targeted Chromatin Ligation, A Robust Epigenetic Profiling Technique for Small Cell Numbers

### INTRODUCTION

For epigenetic investigations of rare cell populations to be routinely performed by researchers of variable skill levels, without expensive and complicated devices and procedures, we have developed a new technique for profiling epigenetic landscapes that enhances sensitivity and simplifies the workflow.

We present a simple, novel, bead-free approach for detecting genome-wide histone modification patterns using targeted chromatin ligation (TCL). Our strategy uses proximity ligation of antibody bound adapter, followed by selective amplification of ligated chromatin to enhance the signal relative to background. Our approach utilizes a simple chromatin fragmentation strategy, eliminates the need for bead-based immunoprecipitation and washing, and purifies all DNA, allowing unligated nucleotides to provide a carrier effect instead of using additional material. The entire procedure has less processing and handling steps, and less hands-on time than conventional ChIP-seq, thus providing greatly reduced methodological complexity while generating improved sensitivity and ease of use.

Material and Methods

Targeted Chromatin Ligations.

Reagents: Chromatin Digestion Buffer (CBD): 33 mM Tris-acetate, pH 7.9, 66 mM potassium acetate, 10 mM magnesium acetate, 0.25% Triton X-100, 1 mM EGTA, 10 mM sodium butyrate. 2× TCL (and N-ChIP) dilution buffer (TDB): (220 mM KCl, 50 mM Tris-acetate, pH 7.9, 0.2% Sarkosyl (Teknova S3376), 0.2% sodium deoxycholate, 1.75% Triton X-100, 40 mM EDTA, 1 mM EGTA). The enzyme mix (EM) used to fragment chromatin contains an equal volume of SaqAI (MseI), FspBI (BfaI), Csp6I, and NdeI from Thermo Fisher (FD2174, FD1764, FD0214, FD0583). A protease Inhibitor (PI) cocktail solution (Roche #4693159001 dissolved in PBS to produce a 20× stock) was added to chromatin digestions.

Antibodies used include: Anti H3K4me3 (Abcam ab8580), anti-H3K27me3 (Active Motif #39155), anti-H3K36me3 (Abcam ab9050), and anti-H3K27ac (Active Motif #39133), and were conjugated with Abcam streptavidin conjugation kit (ab102921). After conjugation, antibodies were concentrated with Pierce concentrator columns (100 MWCO 0.5 ml), then diluted to 1 μg/μl with PBS and final concentrations of 150 mM NaCl and 30% glycerol. To prepare working stocks of Antibody-Adapter complexes, 5 μg of antibody (˜33 pmol) were incubated in 25 μl 1× TCL buffer (equal volumes CBD+TDB) with 41.25 pmol TCL adapters (Table 3, ordered from Integrated DNA Technologies) for 2+ hours at 4° C. Antibody-Adapter stocks were then diluted to 25-50 ng/μl where appropriate, with 1× TCL buffer. We used T4 DNA ligase (EL0011) and Ligation Buffer (Fisher FERB69). Q5 High Fidelity 2× master mix was used for PCR amplification (New England Biolabs M0492). For transposition-based library construction, NEXTERA DNA prep kit (IIlumina FC-121-1031) was used. We also used Axygen beads for purifying/size selecting libraries after indexing (Fisher MAGPCRCLS).

Protocol: Chromatin fragmentation was performed by adding 10 μl of digestion mix (150 μl CDB+8 μl PI+4 μl EM) to the cell pellet (spun down at ˜1000 G for 10 minutes) in 1.7 ml tubes (Axygen MCT-175-C). Cells were resuspended by pipetting ˜10×. Samples were then placed in a water bath for 30 minutes at 37° C. Digestion was stopped by addition of an equal volume of TDB.

3-5 μl of antibody-adapter complex was added to each TCL sample, mixed by pipetting ˜10×, and then samples were placed at 4° C. overnight in a rack without mixing. For MCF7 TCLs, the recommended amounts of antibody bound by adapter are: ˜200 ng anti-H3K27me3, ˜80 ng anti-H3K36me3, ˜40 ng anti-H3K4me3, or ˜100 ng anti-H3K27ac. For neurospheres or other normal mouse cells, the recommended amounts are: ˜80 ng anti-H3K27me3, ˜40 ng anti-H3K36me3, or ˜20 ng anti-H3K4me3). For other cell types/lines, it is recommended to test antibody-adapter amount, beginning with a quantity proportional to the DNA content/cell relative to MCF7 or normal mouse cells.

The next day, samples were placed on the work bench and allowed to reach room temperature (˜15 minutes). 180 μl of ligation mix (1× ligation buffer+1 unit ligase) was then added to each sample and mixed by pipetting 2×, then samples were incubated for 10 minutes at RT. 20 μl of 10% Sarkosyl solution was added to each sample, followed by 10 μl of proteinase K (10 mg/ml). Samples were incubated for 1+ hour at 65° C. to digest protein. DNA was column purified (ZYMO DNA clean and concentrator-5) and eluted in 15 WEB.

The purified TCL DNA was next used in a 60 μl PCR amplification reaction with 2× Q5 polymerase mix (98° C. for 10 s, 63° C. for 30 s, 72° C. for 2 minutes). For TCL reactions with two adapters, ˜15-18 cycles were used. For TCL reactions with a single adapter, ˜25-30 cycles of amplification were used. Single adapter/primer amplifications are ˜40% as efficient as standard PCR, as determined by qPCR, and thus equivalent to ˜15-18 cycles of standard PCR. After amplification, samples were purified with ZYMO columns (30 μl WEB) then quantified with a Qubit 3.0 and HS dsDNA assay kit. Amplifications typically yielded ˜100-700 ng of DNA for 2000 cell TCLs. All TCL samples used in this manuscript were produced using single adapter (A) TCL reactions.

Chromatin Immunoprecipitations. ˜1 million MCF7 cells were resuspended in 0.25 ml CBD+PI+10 μl of enzyme mix. Chromatin Digestions were performed at 37° C. for 30 minutes, followed by dilution with 0.25 ml TDB. Insoluble material was removed by centrifugation at 10,000G for 10 minutes followed by transferring the solubilized chromatin solution to a new tube. Chromatin was then precleared with 50 μl magnetic Protein A-Dynabeads for 2 hours (Invitrogen 10002D). Dynabeads were prepared by washing and resuspension with 1× TCL buffer prior to use. 50 μl of chromatin solution was saved for Input. Another 50 μl Dynabeads, with either 1 μg of anti-H3K36me3, 2 μg of anti-H3K27me3, or 1 μg of anti-H3K27ac, was added to the chromatin solution and then they were incubated overnight at 4° C. with rotation. Bead bound chromatin was washed twice with 1× TCL buffer, once with 1× TCL buffer containing 0.3 M NaCl, and twice with TE. DNA was eluted by resuspending beads in 100 μl TE containing 1% SDS and 10 μg Proteinase K (10 mg/ml), followed by incubation at 65° C. for two hours, with mixing every ˜15 minutes. Beads were removed by magnet and DNA was column purified using Zymo columns and 30 μl elution buffer. ChIP enriched DNA was quantified using a Qubit 3.0 and dsDNA HS assay. N-ChIPs yielded ˜200-300 ng (H3K36me3), ˜30-60 ng (H3K27me3), and ˜60-140 ng DNA (H3K27ac).

For low cell number ChIPs, 200,000 cells were digested as described above, in 0.2 ml digestion volume, and processed identically to generate 0.4 ml of precleared chromatin at 500 cell/W. 10,000, 2000, 400, or 200 cell equivalents were then aliquoted to PCR tubes and diluted to 200 μl with 1× TCL buffer. We used 125 ng anti-H3K36me3, 250 ng anti-H3K27me3, or 125 ng anti-H3K27ac for each ChIP, with 15 μl of beads. After overnight incubation, samples were washed as described above, then eluted in 50 μl TE for PK digestion. After column purification, samples were eluted in 10 μl.

QPCR analysis. Primers were selected for use after being verified to have similar amplification curves across a 10,000-fold range of input, with no amplification in no template controls, prior to conducting any qPCR analysis. ˜20-50 ng of Amplified TCL DNA and ChIP DNA for H3K27me3 samples were analysed by qPCR (10 μl reactions, performed in triplicate) using an AB 7900HT and SYBR Green PCR Master Mix (Applied Biosystems 4309155). SDS software version 2.4 was used to analyse qPCR data. Standard 40 cycle reaction conditions were used (95° C. for 10 s, 60° C. for 10 s, 72° C. for 1 min). Primers (see Table 2) were used at 250 nM. Data was reported as Normalized Fold Signal by first calculating the ratio of input, then normalizing all data points against a chosen negative control region.

Library construction. 25-40 ng of amplified TCL DNA or high cell number N-ChIP enriched DNA were used for library construction using transposition based NEXTERA (followed manufacturer's protocol with ˜8 PCR cycles for indexing). Input samples were made into libraries using NEXTERA with ˜10 cycles of PCR for indexing. Libraries for low cell number ChIP samples (and input) generated from 10,000-200 cells were made using NEXTERA XT (followed manufacturer's protocol with 14 cycles of PCR for indexing). Indexed samples were quantified by Qubit 3.0, then pooled to produce ˜5 nM samples ready for submission to sequencing facilities. TCL and N-ChIP libraries were sequenced on a NextSeq500 to obtain 75 bp single end reads with read depths of ˜30-60 million reads.

Cell Preparation. MCF7 (ATCC HTB-22) cells were cultured in DMEM supplemented with 10% fetal bovine serum and penicillin-streptomycin-glutamine. Cells were trypsinized, pelleted, washed 2× with PBS, then resuspended and counted by hemocytometer. For TCLs, cells were then diluted to 100 cells/μl and 2000 cells were aliquot to 1.7 ml tubes containing 200 μl PBS. Cells were then pelleted at ˜1000G for 5-10 minutes for TCL. For TCLs with 10,000-200 cells, after counting cells, they were diluted to 500 cells/μl, then serially diluted to 100 cells/μl, and 20 cells/μl, prior to making the 10,000-200 cell aliquots pelleted for TCL.

To produce neurospheres, mice (Black6 from a mixed C57B16 and B6C3 background) were euthanized by CO₂, decapitated, and their brains immediately removed. The subventricular zone (SVZ) was micro-dissected and stored in ice-cold PBS for further processing. The tissue was digested using Liberase DH (Roche) and DNase I (250 U ml⁻¹) at 37° C. for 20 minutes followed by trituration. Digested tissue was washed in ice-cold HBSS without calcium and magnesium, filtered through a 40-μm filter and FACS-sorted as Lineage⁻CD24⁻ cells into neurosphere growth media that is, Neurobasal-A (Invitrogen) supplemented with Glutamax (Life Technologies), 2% B27-A (Invitrogen), mouse recombinant epidermal growth factor (EGF; 20 ng ml⁻¹) and basic fibroblast growth factor (bFGF; 20 ng ml⁻¹) (Shenandoah Biotechnology). Lineage cells were depleted using mouse CD45, CD31, and Ter119 (Biolegend).

After neurospheres formed, they were FACS-sorted into CD15⁺Egfr⁺ and CD15⁻Egfr⁺ or CD15⁻Egfr⁻ cells. In total, approximately 7,000 cells were sorted for each population before individually processing 1000 sorted events/cells for each TCL, as described for MCF7 cells. For FACS analysis the cells were stained with anti-CD15-fluorescein isothiocyanate (FITC) (MMA; BD), and EGF complexed with Alexa647-streptavidin (Life Technologies).

ENCODE Data. MCF7 aligned reads from GEO data sets GSM945854, GSM970218, GSM970217, and GSM945859 were downloaded from genome.ucsc. edu/cgi-bin/hgTrackUi?db=hg19&g=wgEncodeSydhHistone.

Sequence analysis. Raw sequence reads were uploaded to Galaxy (usegalaxy.org) and aligned to the human genome (hg19) or mouse genome (mm9) using Bowtie (−1,15; −e, 40; −v, 2; −m, 1) and Bowtie2 (-very-fast-local), respectively. Only uniquely mapped reads were retained for further analysis. Alignment files were used to produce signal tracks with DeepTools (100 bp bins with 500 bp read extensions and RPKM normalization). We also filtered out ENCODE blacklisted regions (downloaded from sites.google.com/site/anshulkundaje/projects/blacklists). Resulting signal files were used for Principal Component Analysis and correlation analysis. Signal files were loaded into the Broad Institute's IGV browser to visualize data. The neurosphere H3K4me3 data were further filtered to show only reads within promoters (defined as 5,000 bp around TSSs based on RefSeq). To calculate fraction of reads in peaks, we used MACS2 (-nomodel, p=0.01, -broad, cuttoff 0.1, duplicates=auto, extension 200) to call peaks using ENCODE MCF7 ChIP data. The resulting Broad Peak BED files were used with Samtools to extract all reads located within peak regions and compared read counts to those of the unfiltered alignment files. Cross-correlation plots were generated for de-duplicated BAM files using Phantompeakqualtools (Marinov et al. (2014) G3 (Bethesda) 4(2):209-223), with strand shifts ranging from 0 to 1000 bp at a step size of 5 bp, and otherwise default parameters. We employed ngs.plot (Shen et al. (2014) BMC Genomics 15:284) with default parameters to generate aggregation plots across all TSS intervals in the hg19 reference genome.

Availability of sequence data. All raw data (FASTQ) files and signal files (BigWig) files used for the production of this manuscript have been deposited into the Sequence Read Archive (SRA) and are available through the Gene Expression Omnibus accession number GSE94804.

Results

Development of Targeted Chromatin Ligation

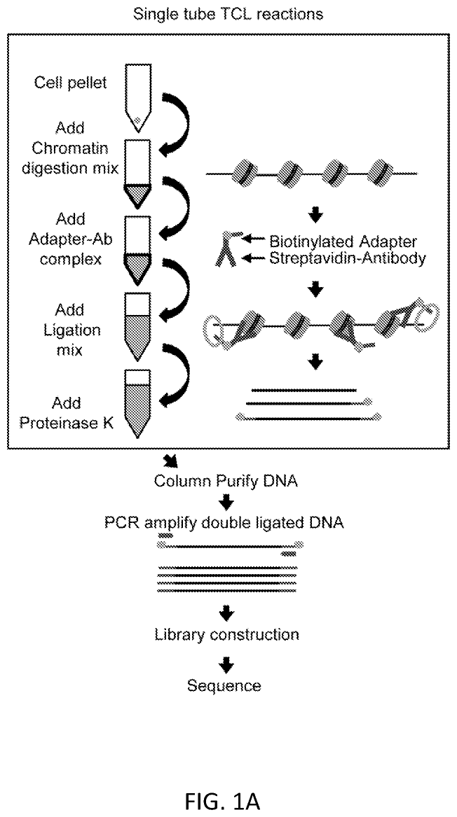

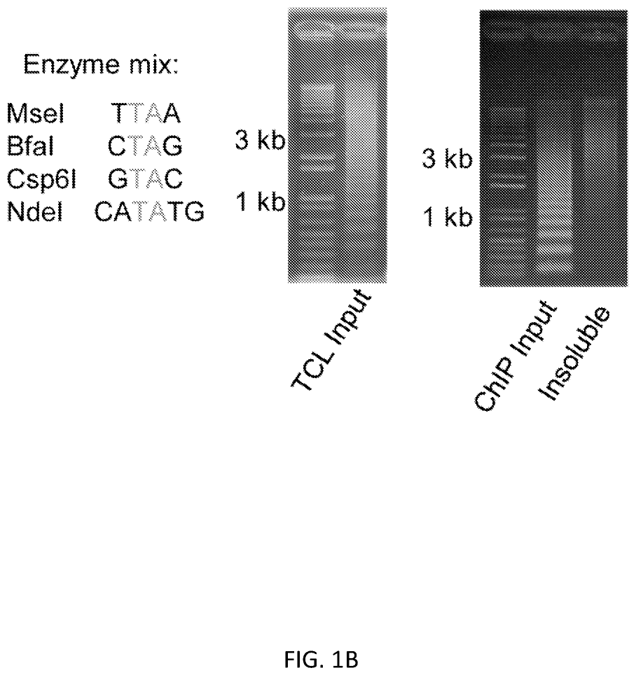

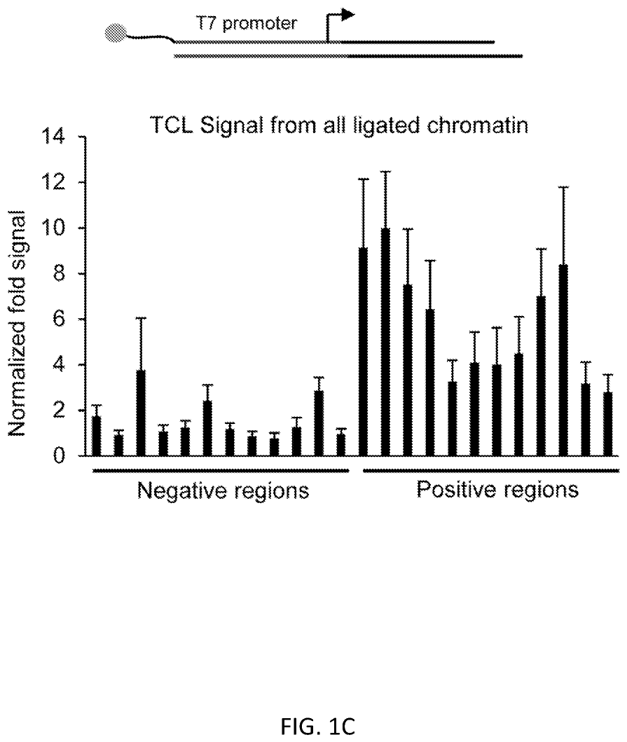

Development of the TCL procedure (FIG. 1A) began with determining suitable chromatin digestion conditions to produce a broad DNA ladder. ChIP-seq techniques utilize one of two strategies to fragment chromatin, sonication or enzymatic digestion with micrococcal nuclease. Sonication is the preferred method when using fixed chromatin, but requires a larger working volume that increases loss of material through greater absorption and destroys some epitopes, contributing to loss of material and limited sensitivity of the assay. Micrococcal nuclease is the preferred method when working with native chromatin, but as with sonication, the ends of the chromatin are not uniform and require processing that is inefficient and laborious. Additionally, careful titration of micrococcal nuclease is required to prevent over digestion. We sought to simplify the chromatin preparation step so that the experimenter could avoid loss of material, eliminate the need for sonication equipment, and easily avoid excessive digestion. We therefore decided to use a cocktail of restriction enzymes (three 4-base cutters and one 6-base cutter) that produce identical dinucleotide overhangs. Such overhangs can be efficiently ligated relative to the blunt ends or single nucleotide overhangs produced by processing chromatin fragmented by sonication or micrococcal nuclease. We digested unfixed cells in a 10 μl volume of buffer that permeabilizes the cells, followed by a two-fold dilution in buffer that terminates digestion with EDTA and lyses the cell. Since our TCL procedure does not use beads, we do not need to pellet debris and transfer material to new tubes, a step required by ChIP procedures that reduces background, but contributes to material loss. After testing digestion conditions, we were able to generate consistent DNA ladders for TCL and ChIP (FIG. 1B).

After determining suitable chromatin digestion conditions, we proceeded to test various reaction parameters of targeted chromatin ligations: antibody concentrations, adapter concentrations, salt concentrations, and ligation conditions (volume, temperature, ligase amount). We used 2,000 MCF7 cells for each TCL reaction during testing, and all initial testing used anti-H3K27me3 antibody conjugated to streptavidin. After chromatin fragmentation, 3-5 μl of streptavidin-conjugated antibody with bound adapter was added to the 20 μl fragmented chromatin solution, then incubated overnight (FIG. 1A). As rotation or agitation of dilute small volume samples could contribute to significant loss of material through absorption to surfaces, we incubated TCL samples without mixing during the overnight incubation. The next day, 180 μl of a ligation mix was added to samples to allow adapters to ligate to chromatin ends. We found the most important parameters for successful TCL reactions to be a 1:1 adapter to antibody ratio and PCR selection of only double ligated chromatin.

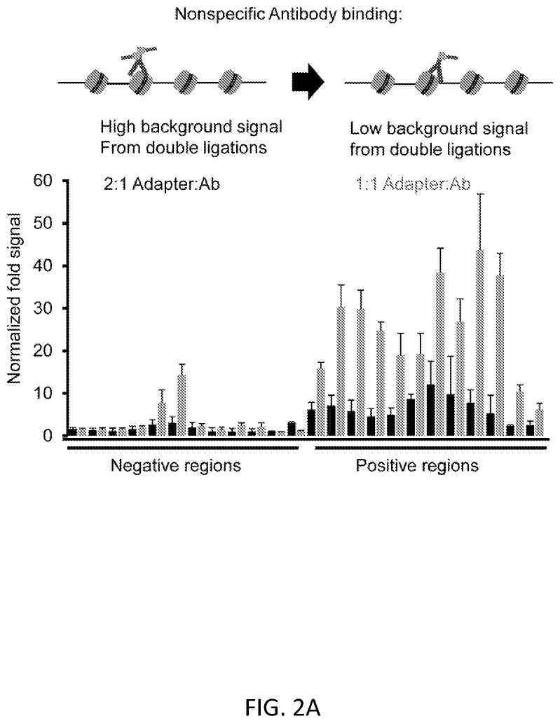

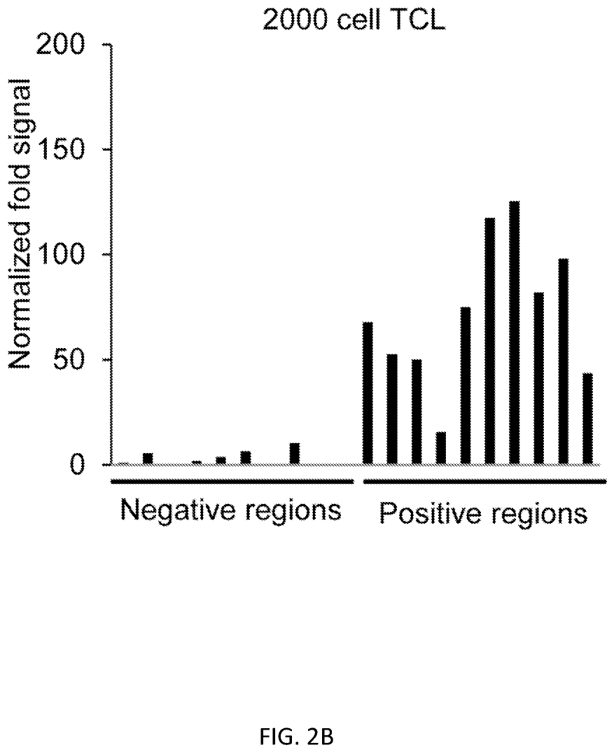

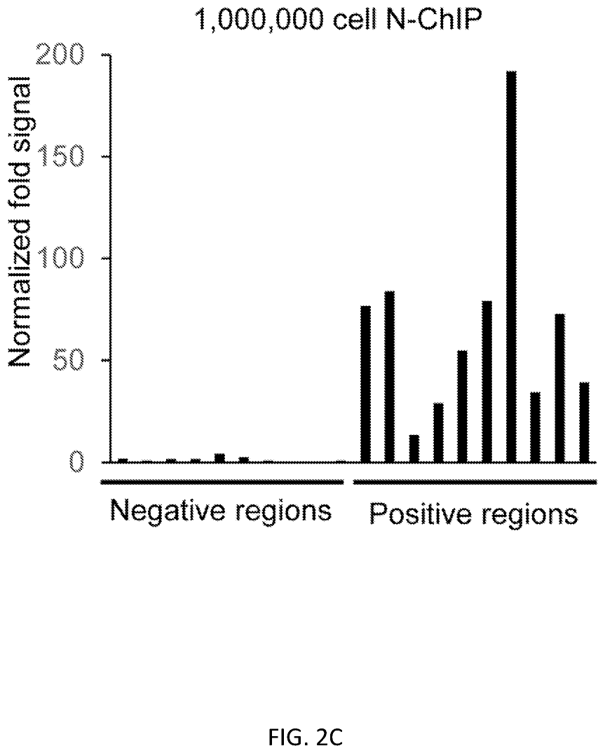

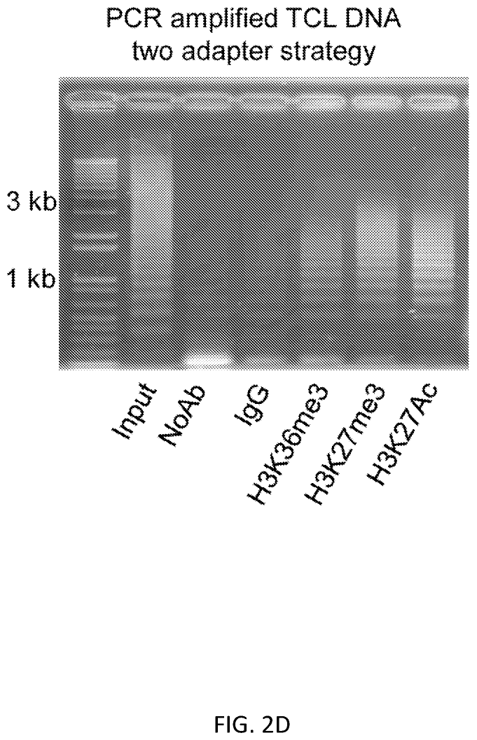

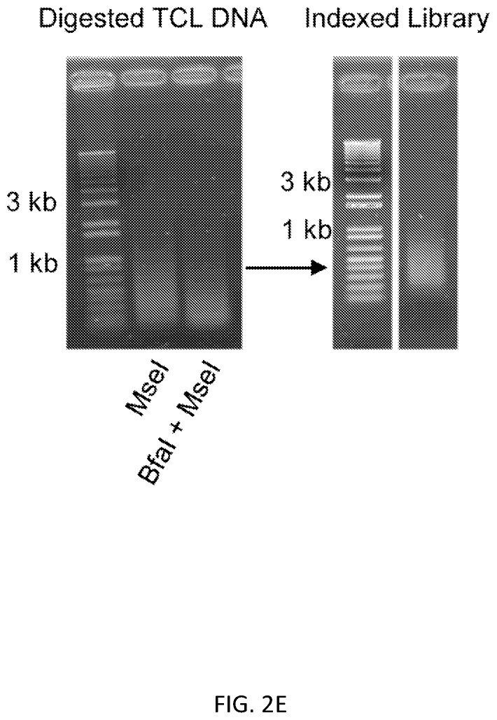

Initially, we considered that most ligated chromatin fragments might be ligated at only one end, so a T7 promoter was included on the adapters. TCL reactions were then analysed by T7 based RNA amplification, followed by reverse transcription and qPCR. When we tested various reaction conditions, we observed highly robust yet limited signal that was insensitive to antibody and adapter concentrations (FIG. 1C), or other conditions tested (data not shown). The importance of using a low amount of adapter became clear when we examined only double ligated chromatin amplified by PCR (FIG. 2A). Combining selection for double ligations and reducing the ratio of adapter to antibody to 1:1 increased signal to noise detected by TCL-qPCR and made signal sensitive to antibody titration. To evaluate these optimizations, we compared the performance of TCL-qPCR using only 2,000 cells with native chromatin ChIPs (N-ChIP) performed with one million cells and identical reagents as TCL. Both methods yielded comparable signals (FIGS. 2B and 2C).

Adapter and Library Construction Considerations

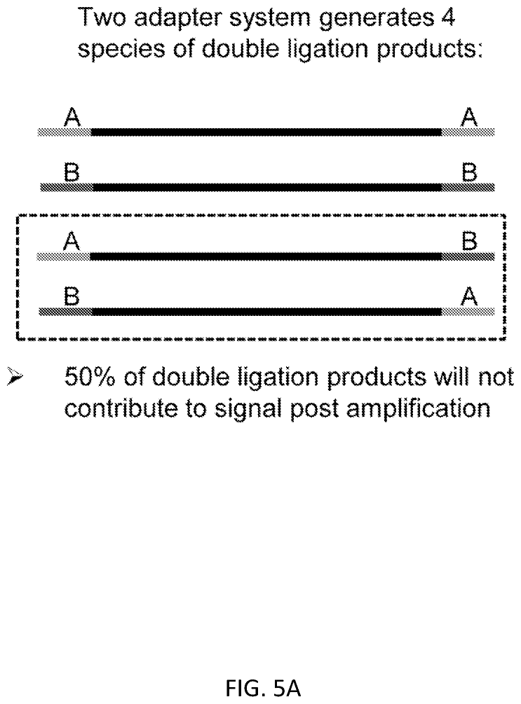

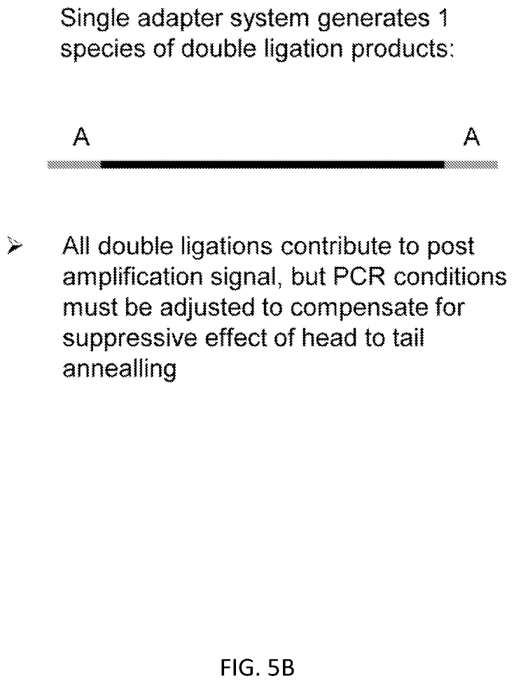

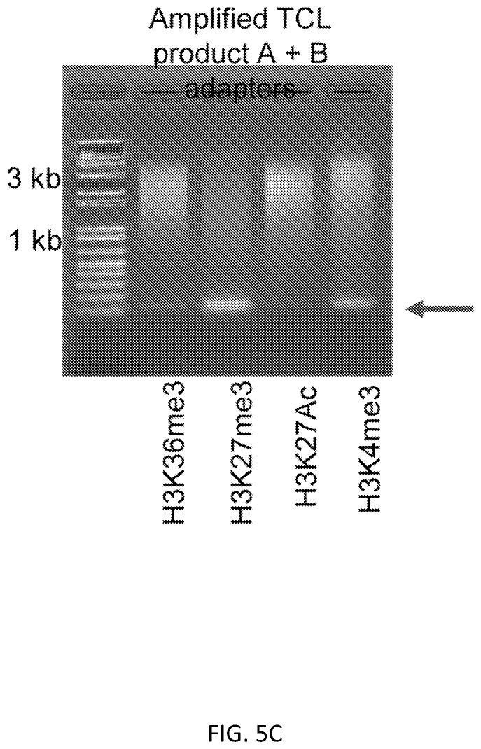

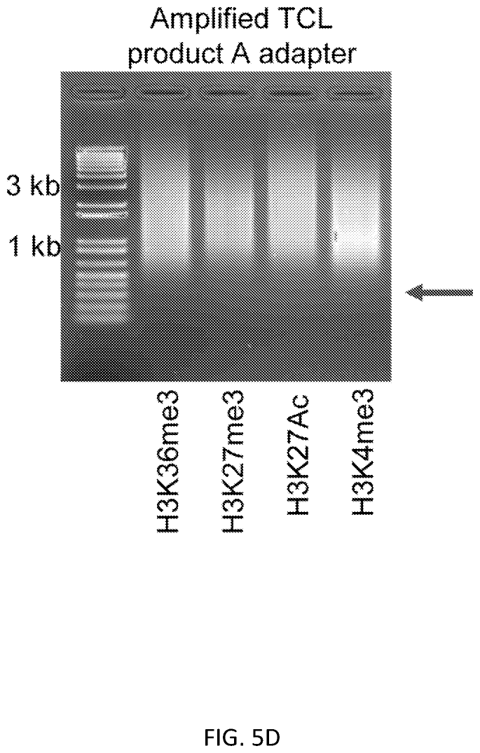

During the initial development of TCL, we performed reactions using antibodies loaded with two adapters, an “A” adapter and a “B” adapter. We were concerned that using two adapters would limit sensitivity when nearing the lower limit of input cell numbers for TCL reactions. Two adapters lead to formation of four species of double ligation, but PCR amplification would only efficiently amplify half those species of ligation products, potentially leading to drop out of signal during amplification (FIGS. 5A-5B). We therefore switched to using a single adapter for TCL reactions and found that one adapter is preferable, in part due to elimination of PCR generated primer dimer artefacts (FIGS. 5C-5D, arrows). However, as head to tail annealing of denatured double ligated DNA having the same adapter on either end suppressed PCR amplification efficiency (˜35-45% efficiency based on qPCR, data not shown), we needed to increase the number of PCR cycles during amplification to compensate.

Having validated TCL reactions with either one or two adapters by qPCR, we proceeded with library construction to evaluate genome-wide histone profiles. PCR amplified TCL DNA (FIGS. 5C-5D) requires further fragmentation to produce next generation sequencing libraries. We used transposition based tagmentation that simultaneously fragments DNA while inserting sequencing adapters, to construct sequencing libraries.

Genome-Wide Analysis of TCL Versus ChIP

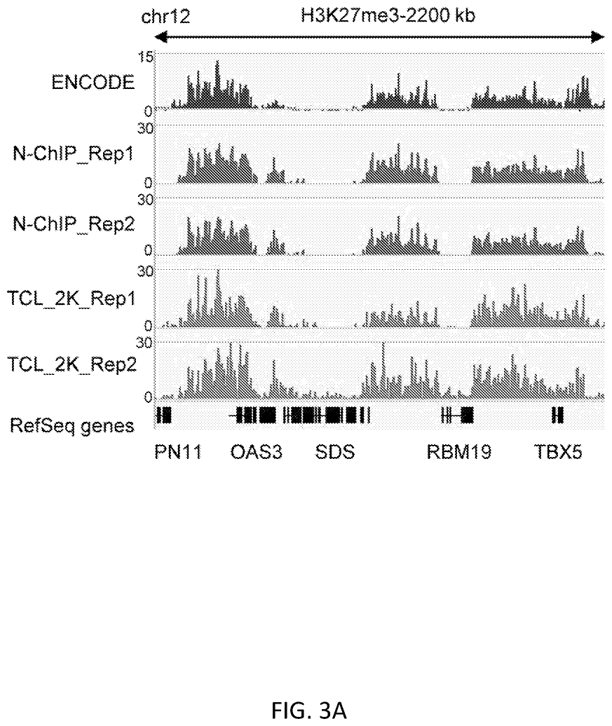

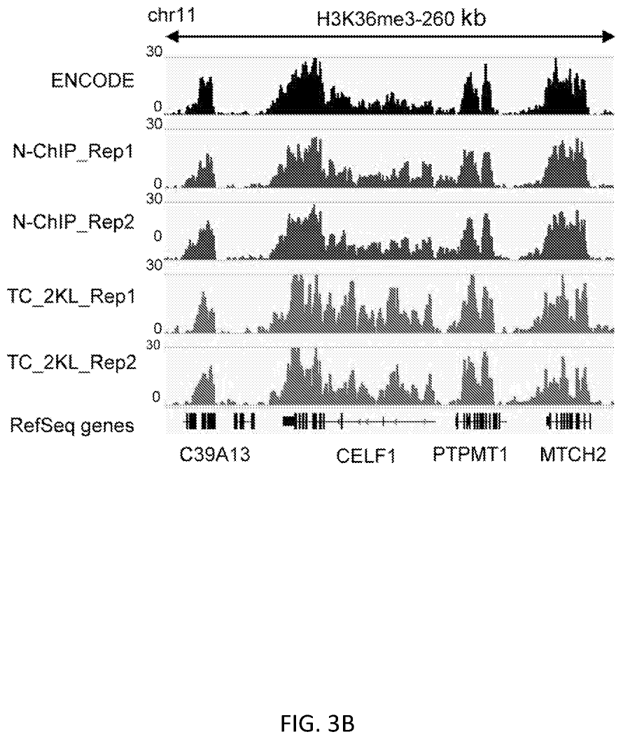

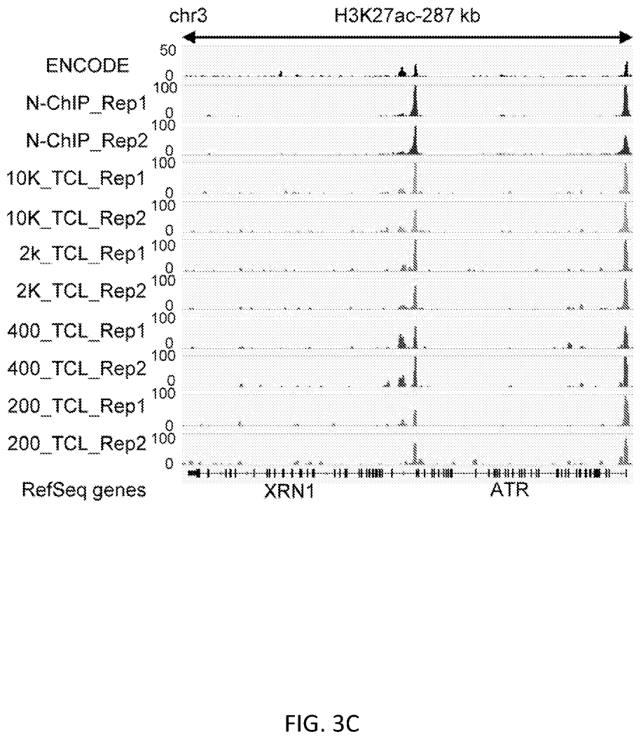

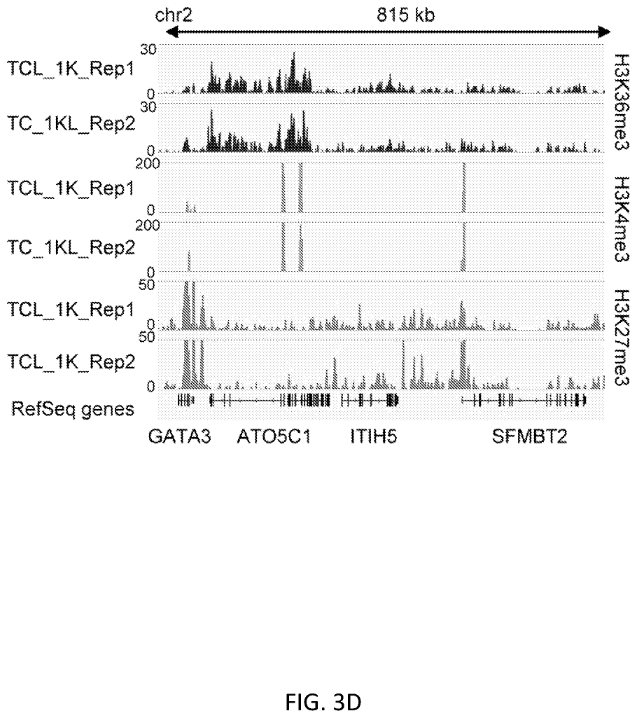

After sequencing, we downloaded ENCODE data for MCF7 (Dunham et al. (2012) Nature 489:57-74) and analysed all libraries using Galaxy. We then performed both qualitative and quantitative assessments of TCL performance. First, we visually examined genome-wide data quality using normalized signal tracks. When comparing data generated from 2,000 cell TCLs and 2000 cell ChIPs to ChIP samples produced with a million or more cells, H3K27me3 chromatin profiles generated with TCL were virtually indistinguishable across a range of genomic intervals (FIG. 3A). Genomic windows comparing H3K36me3 marks between 2,000 cell TCLs and ChIP were also highly concordant (FIG. 3B). While 2000 cell ChIPs were able to produce signal reminiscent of one million cell ChIPs and 2000 cell TCLs, the signal was clearly reduced (FIGS. 3A and 3B). To further evaluate the sensitivity of TCL, we then compared H3K27ac chromatin landscapes produced with high cell number ChIPs to TCL and ChIP data generated with decreasing cell numbers (10,000-200 cells). Strikingly, the TCL signal was consistently retained even as the number of cells used was reduced to 200, while the ChIP signal dropped sharply even at 10,000 cells, and continued to decrease with less cells (FIG. 3C). While we did not perform N-ChIPs for H3K4me3 and there is no equivalent ENCODE data for that histone mark to make a comparison, we did generate highly reproducible 2,000 MCF7 cell TCLs for H3K4me3; visual examination relative to other histone marks revealed high feature specificity with clearly reproducible peak patterns. Since MCF7 is a human cancer cell line with an abnormal karyotype and high DNA content (˜10 pg/cell), we sought to test TCL on a normal low DNA content (˜5 pg/cell) cell type. We also sought to ensure that TCL works in the context of sorting limited cell numbers. To accomplish this, we sorted mouse brain derived neurosphere cells and performed TCLs on ˜1,000 sorted events (<1,000 actual cells). The resulting epigenetic profiles were highly concordant (FIG. 3D).

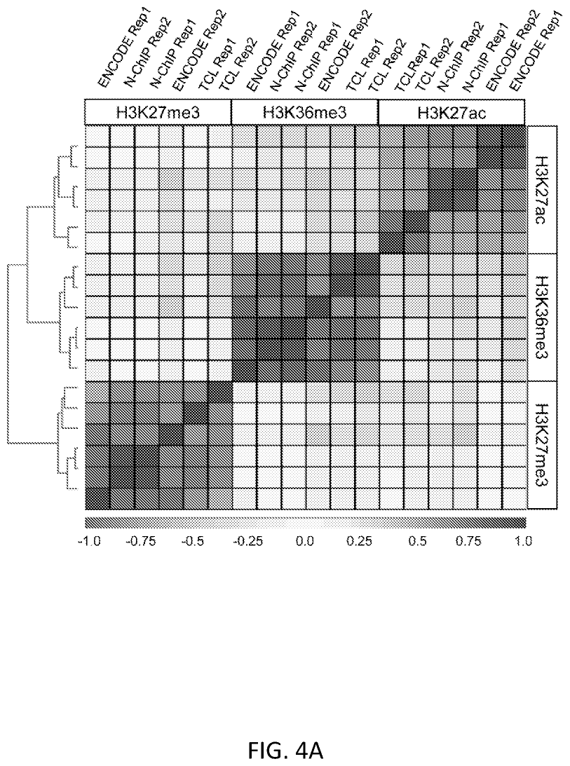

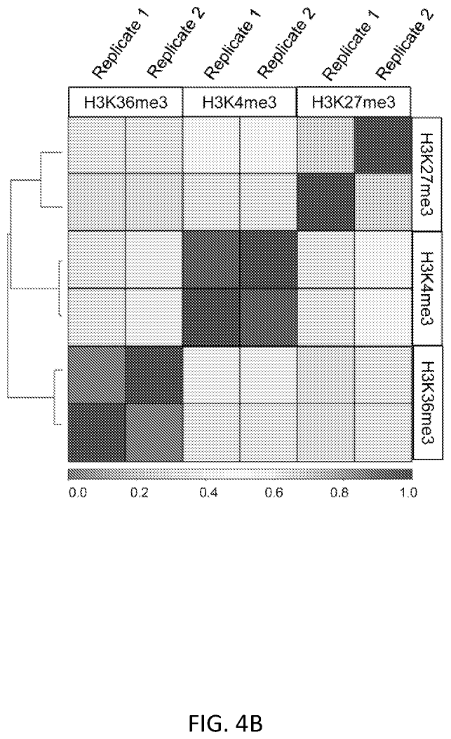

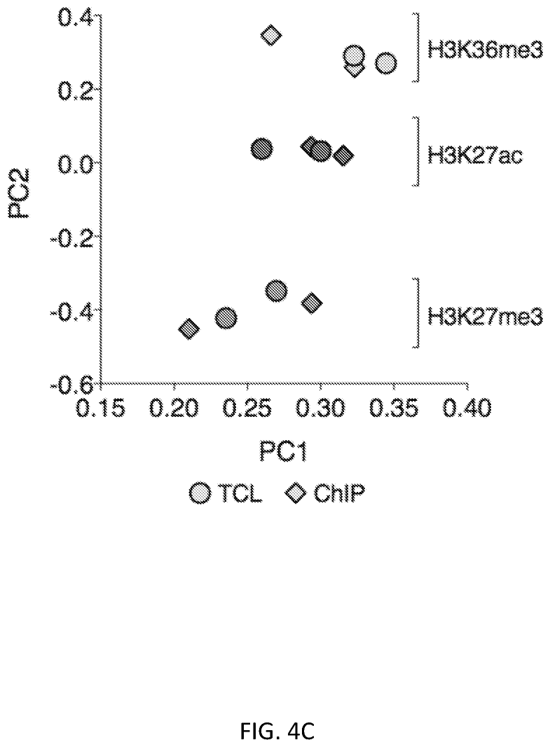

To quantitatively evaluate the robustness of genome wide TCL data, both across biological replicates and in comparison, to ChIP data, we used several approaches, including: examination of signal to noise represented by the fraction of reads in peaks (FRIP, Landt et al. (2012) Genome Res. 9:1813-1831, Kellis et al. (2014) Proc. Natl. Acad. Sci. U.S.A. 111(17):6131-6138), correlation analysis, and principal component analysis (PCA). First, we calculated the FRIP for all MCF7 data profiled by both TCL and ChIP, and found that low cell number TCL samples had superior FRIPs when compared to low cell number ChIPs, and nearly equivalent FRIPs compared to ENCODE ChIPs (Table 1). Notably, the FRIPs generated with 200 and 400 cell H3K27ac TCLs averaged 25.1% and 30.5%, respectively, compared to ENCODE ChIPs that averaged 27.1% (Table 1). In contrast, ChIP samples produced with 2000, 400, or 200 cells had FRIP scores 3-9% less than comparable TCLs, consistent with the visually inferior signal to noise observed (FIGS. 3A-3C). We also evaluated signal to noise ratios by strand cross-correlation analysis (Landt et al. (2012) Genome Res. 9:1813-1831, Marinov et al. (2014) G3 (Bethesda) 4(2):209-223) and found the results to be consistent with FRIP, as low cell TCLs had higher normalized strand coefficients (NSCs) and higher relative strand correlations (RSC) compared to equivalent cell number ChIPs. Aggregation plots (Shen et al. (2014) BMC Genomics 15:284) of H3K27ac around TSS intervals also suggest that TCLs are more similar to one million cell N-ChIPs than low cell N-ChIPs. Next, we produced genome-wide Pearson correlation plots using 2 kb genomic windows. Strong correlations between TCL and ChIP data were observed (FIG. 4A). Genome-wide correlations for TCL versus high cell number N-ChIP averaged r=0.69 for H3K27me3, r=0.83 for H3K36me3, and r=0.6 for H3K27ac. Correlations between biological replicates of TCLs were high and comparable to ChIPs, with nearly all replicates having r>0.8 using 2 kb genomic bins (FIG. 4A). Biological replicates of 2000 cell H3K4me3 TCLs were also highly correlated with r=0.88. Additionally, analysis of the 10,000-200 cell H3K27ac TCLs demonstrated high correlations between biological replicates and comparing 10,000 cell TCLs to 400 cell TCLs revealed high correlations. Genome wide correlations for the <1000 cell neurosphere TCLs were also very high with r=0.71-0.97 (FIG. 4B). Finally, we performed PCA analysis on the TCL and ChIP data from MCF7 samples using various bin sizes, from 500 bp to 20 kb, and found that the TCL data and ChIP data clustered together based on histone marks. Increasing the bin size had a negligible effect as data was already well clustered using small 500 bp windows (FIG. 4C). Notably, the TCL samples appeared to cluster more tightly than the ChIP samples, indicating strong reproducibility (FIG. 4C).

Discussion

The simple workflow of the TCL technique, outlined in FIG. 1, begins with resuspension of unfixed cells in digestion buffer containing enzymes that generate native chromatin fragments with identical dinucleotide overhangs. While ChIP-seq methods seek to fragment chromatin into small fragments (250-500 bp), which reduces background chromatin binding to beads, facilitates library construction, and maximizes data resolution, our bead-free strategy and library construction method obviate the need for small fragments and allows the strategic use of larger chromatin fragments for greater sensitivity.

The large chromatin fragments facilitate relatively symmetrically distributed background binding events, presumably to unmodified histones, that likely drives most ligation events detected in FIG. 1C. Since our technique eliminates washing to improve stringency, and most background appears to be driven by background antibody binding and not reaction parameters, signal specificity was limited when examining all ligation events. We took advantage of the apparent background binding by hypothesizing that if the initial ligation of chromatin ends is driven by specific or nonspecific antibody binding, secondary ligation events should be driven by higher locally concentrated adapters as a function of antibody specificity and concentration. Therefore, using enough antibody to have at least one nonspecific binding event per chromatin fragment, but not two, should facilitate capturing more frequent specificity driven double ligations when limiting the amount of adapter bound to the antibody, and producing increased signal to noise while also maintaining sufficient depth of coverage for regions with lower abundancy of modified histone targets across multinucleosome fragments that might fail to produce double ligations without nonspecific binding events. Since double ligations should occur more frequently with bigger fragments that can support both specific and nonspecific antibody binding, amplification of double ligated chromatin should select for larger fragments. The data presented in FIG. 2D supports this interpretation and shows that ligations in the presence of IgG or no antibody selects smaller fragments where ligations are driven by diffusion and intermolecular ligation, not intramolecular proximity ligation.

As cell numbers used for epigenetic profiling decrease, it may be expected that methods will become increasingly sensitive to antibody quality. For example, we originally tested three ChIP-seq validated antibodies against H3K27me3, including Millipore #07-499, used by ENCODE, and found all three worked well for high cell number ChIP. Indeed, all three produced similar results for TCL-qPCR, but one produced sequence data that better matched ENCODE H3K27me3 signal tracks (data not shown). Thus, researchers should not assume all ChIP validated antibodies are compatible with TCL or any other low cell genome wide profiling technique.

While amplification of ligated material prior to transposase based library construction masks the duplication rate, and single end reads do not support accurate estimation of duplication rates, we did assess duplication rates and found that ˜17-27% of unique reads map to identical 5′ sequences. That likely overestimates the real duplication rate, suggesting the library complexity produced by TCL remains high, and is superior to other low cell epigenetic profiling techniques. For example, the single end data from ChIP-seq with 10,000-100 cells, produced by a microfluidic ChIP-seq device (Brind'Amour et al., supra), had duplication rates calculated to be in the range of 55-80%. The apparent low duplication, along with the robustness demonstrated by our principal component analysis and correlation analysis clearly indicate that TCL produces high quality robust data.

We have demonstrated a greatly simplified approach for producing high quality histone modification profiles that is unique and distinct from ChIP. Key advantages of TCL include greatly reduced handling through elimination of inefficient immunoprecipitation, washing, and the subsequent need for inefficient enzymatic end repair and single nucleotide or blunt-end ligation steps with picogram quantities of starting material. These qualities should make TCL more amenable to microfluidic adaptation, automation, and further optimization with even less than the 200 cells tested here. While the current iteration of TCL was designed and tested only for mapping histone modifications, we are currently working to adapt TCL for use with transcription factors. We also believe TCL offers the opportunity for studies beyond what is possible for ChIP, such as multiplexing of histone modifications or transcription factor co-occupancy without re-ChIP. For example, it may be possible to preload antibodies against H3K27me3 and H3K4me3 with different barcoded adapters so that their simultaneous use can allow direct amplification and detection of true bivalent chromatin. TCL thus provides robust epigenetic profiles from low cell numbers in an easy to execute approach with the potential for novel applications.

TABLE 1
Fraction of reads in peaks (FRIP) were calculated for MCF7
TCL-seq and ChIP-seq data for H3K36me3, H3K27me3, and H3K27ac
histone marks. Peaks were called using MACS2 peak calling parameters
suitable for broad peaks, as recommended by ENCODE. Identical
peak calling parameters were used for all samples.

| | All Unique | Unique reads | |
|---|---|---|---|
| Sample | reads | in Peaks | FRIP |
| TCL-2K-H3K36me3-Rep1 | 60930711 | 22177299 | 0.363975713 |
| TCL-2K-H3K36me3-Rep2 | 33857568 | 12766582 | 0.377067307 |
| N-ChIP-H3K36me3-Rep1 | 41255535 | 19746921 | 0.47864901 |
| N-ChIP-H3K36me3-Rep2 | 38408827 | 19743023 | 0.514023066 |
| ENCODE MCF7- | 25318535 | 11712177 | 0.462592998 |
| H3K36me3 Rep1 | | | |
| ENCODE MCF7- | 28802418 | 11351045 | 0.394100419 |
| H3K36me3 Rep2 | | | |
| TCL-2K-H3K27me3-Rep1 | 30051645 | 9411695 | 0.31318402 |
| TCL-2K-H3K27me3-Rep2 | 29413373 | 9023207 | 0.306772263 |
| N-ChIP-H3K27me3-Rep1 | 40852490 | 15360755 | 0.376005355 |
| N-ChIP-H3K27me3-Rep2 | 27563539 | 10170374 | 0.368979252 |
| ENCODE MCF7- | 34594045 | 11238011 | 0.324853916 |
| H3K27me3 Rep1 | | | |
| ENCODE MCF7- | 31179986 | 8899182 | 0.285413278 |
| H3K27me3 Rep2 | | | |
| TCL-10K-H3K27Ac-Rep1 | 24972549 | 6167649 | 0.246977151 |
| TCL-10K-H3K27Ac-Rep2 | 23781641 | 5587899 | 0.234966923 |
| TCL-2K-H3K27Ac-Rep1 | 25766600 | 7498525 | 0.291017247 |
| TCL-2K-H3K27Ac-2-Rep2 | 17713297 | 4300254 | 0.242769824 |
| TCL-400-H3K27Ac-Rep1 | 26458816 | 7917526 | 0.299239618 |
| TCL-400-H3K27Ac-Rep2 | 35314640 | 10772197 | 0.305034881 |
| TCL-200-H3K27Ac-Rep1 | 26839135 | 7018071 | 0.261486482 |
| TCL-200-H3K27Ac-Rep2 | 23096721 | 5112651 | 0.221358304 |
| N-ChIP-H3K27Ac-Rep1 | 35190909 | 16799890 | 0.477392897 |
| N-ChIP-H3K27Ac-Rep2 | 29073330 | 11012318 | 0.378777319 |
| ENCODE MCF7- | 25799447 | 7325880 | 0.283954924 |
| H3K27Ac Rep1 | | | |
| ENCODE MCF7- | 23868427 | 6138221 | 0.257169063 |
| H3K27Ac Rep2 | | | |

TABLE 2
Primers used for qPCR analysis of MCF7 TCLs and N-ChIPs

| | | | Forward | Reverse |
|---|---|---|---|---|
| Negative | P1 | ORC4-1 | CAGCTCTCCTCCTCCTCCTT | AGTCAGAGCCAGAGGAAGGC |
| | | | (SEQ ID NO: 1) | (SEQ ID NO: 2) |
| Negative | P2 | ORC4-2 | TGTTACGTGGTGCCATGACT | AAGGCCAAGGAGTTTGAAGA |
| | | | (SEQ ID NO: 3) | (SEQ ID NO: 4) |
| Negative | P3 | SKAP2-1 | AAGAAGGCCTTTTCCCTTCA | TTAGCATTGGCATCTGCAAG |
| | | | (SEQ ID NO: 5) | (SEQ ID NO: 6) |
| Negative | P4 | SKAP2-2 | CACTAACCTCCCTCCCCTTC | TGGGGTAGCTGTCACCAAAT |
| | | | (SEQ ID NO: 7) | (SEQ ID NO: 8) |
| Negative | P5 | ELF5-1 | AGCTAAGGGATCCAGAGGGA | GTCTGCTCGTCCATCTCCAG |
| | | | (SEQ ID NO: 9) | (SEQ ID NO: 10) |
| Negative | P6 | ELF5-2 | GTGCGTTTGGTGAGGAATTT | TGGGCCTATAATGCTCTTCC |
| | | | (SEQ ID NO: 11) | (SEQ ID NO: 12) |
| Negative | P7 | RPL19-1 | GTGCTGGCATCTATGCTGAA | TTTGCACACAGGCAGAAAAC |
| | | | (SEQ ID NO: 13) | (SEQ ID NO: 14) |
| Negative | P8 | RPL19-2 | GATGAAAGAACCGGACAGGA | CAAGACCGACAGTCCCTTGT |
| | | | (SEQ ID NO: 15) | (SEQ ID NO: 16) |
| Negative | P9 | ATXN3-1 | CCACGTCCAGCTACTCTGGT | GGCCCAAGCATTTCCTTTAT |
| | | | (SEQ ID NO: 17) | (SEQ ID NO: 18) |
| Negative | P10 | ATXN3-2 | AC CAGGAGGGCAATATACCA | TTTCTTGCTGGGTCAATAGGA |
| | | | (SEQ ID NO: 19) | (SEQ ID NO: 20) |
| Negative | P11 | USP25-1 | CGTGTGGGAAGCAGAGTGTA | TCGGTTCAGAAAGGATGACA |
| | | | (SEQ ID NO: 21) | (SEQ ID NO: 22) |
| Negative | P12 | USP25-2 | CTCCTCATGGCAGGTTGTTT | TCATTCGATTCGTCCCTCTC |
| | | | (SEQ ID NO: 23) | (SEQ ID NO: 24) |
| Positive | P13 | ZEB2-1 | GCCTCCTTCTCCTTTGCTTT | CCAGGAACCTAGAATGGCAC |
| | | | (SEQ ID NO: 25) | (SEQ ID NO: 26) |
| Positive | P14 | ZEB2-2 | GCCAGGATCCCTCTATTTCC | CAAGGCTCCCAGAAGTGTTC |
| | | | (SEQ ID NO: 27) | (SEQ ID NO: 28) |
| Positive | P15 | HOXA1-1 | CGCGTCAGGTACTTGTTGAA | ACATTTCCGTCTCATGGCTT |
| | | | (SEQ ID NO: 29) | (SEQ ID NO: 30) |
| Positive | P16 | HOXA1-2 | CGCACGACTGGAAAGTTGTA | CCCATGGAGGAAGTGAGAAA |
| | | | (SEQ ID NO: 31) | (SEQ ID NO: 32) |
| Positive | P17 | EHF-1 | ATTCAGCCATCCAGACAACC | ATCCTCTTCTCTCCGGCAAC |
| | | | (SEQ ID NO: 33) | (SEQ ID NO: 34) |
| Positive | P18 | EHF-2 | TAGCGATCTGGAAACAGGCT | GGGCCTGTTTGGGTTTATTT |
| | | | (SEQ ID NO: 35) | (SEQ ID NO: 36) |
| Positive | P19 | SLC24A4-1 | TGATGATGTGGTTTGCCCTA | CCATGCTTTCACCAAATCCT |
| | | | (SEQ ID NO: 37) | (SEQ ID NO: 38) |
| Positive | P20 | SLC24A4-2 | AGCGGGTTCTGATGTCAATC | TCACGAATGGACACACCAGT |
| | | | (SEQ ID NO: 39) | (SEQ ID NO: 40) |
| Positive | P21 | SKAP1-1 | TCTTTTCCTTGCACCTTGCT | GGGCATGTTGACCAGAGACT |
| | | | (SEQ ID NO: 41) | (SEQ ID NO: 42) |
| Positive | P22 | SKAP1-2 | CACTAACCTCCCTCCCCTTC | TGGGGTAGCTGTCACCAAAT |
| | | | (SEQ ID NO: 43) | (SEQ ID NO: 44) |
| Positive | P23 | GREK1-1 | CTTGCTGAAATGTGGCAGAA | TGGATGTACTTGCCCCAGAT |
| | | | (SEQ ID NO: 45) | (SEQ ID NO: 46) |
| Positive | P24 | GREK1-2 | AAGTCGGAGGAGGGAGAAAG | ATCCACCATCGGATCTCTTG |
| | | | (SEQ ID NO: 47) | (SEQ ID NO: 48) |

TABLE 3
Adapter and IIlumina compatible indexing primers used for Tas and library construction.

| | | |
|---|---|---|
| TCL-A-For | Biotin-T7-Adapter-A | ATAATACGACTCACTATAGGGGTCGTCGGCAGCGTCAGATGTGTATAAGAGACAG |
| | | (SEQ ID NO: 49) |
| TCL-A-Rev | TA-Adapter-A-Reverse | TACTGTCTCTTATACACATCTGACGCTGCCGACGACCCCTATAGTGAGTCGTATTAT |
| | | (SEQ ID NO: 50) |
| | Index Primer A | AATGATACGGCGACCACCGAGATCTACAC[i5]TCGTCGGCAGCGTC |
| | | (SEQ ID NO: 51) |
| TCL-NIP-A501 | Index Primer A501 | AATGATACGGCGACCACCGAGATCTACACTAGATCGCTCGTCGGCAGCGTC |
| | | (SEQ ID NO: 52) |
| TCL-NIP-A502 | Index Primer A502 | AATGATACGGCGACCACCGAGATCTACACCTCTCTATTCGTCGGCAGCGTC |
| | | (SEQ ID NO: 53) |
| TCL-NIP-A503 | Index Primer A503 | AATGATACGGCGACCACCGAGATCTACACTATCCTCTTCGTCGGCAGCGTC |
| | | (SEQ ID NO: 54) |
| TCL-NIP-A504 | Index Primer A504 | AATGATACGGCGACCACCGAGATCTACACAGAGTAGATCGTCGGCAGCGTC |
| | | (SEQ ID NO: 55) |
| TCL-NIP-A505 | Index Primer A505 | AATGATACGGCGACCACCGAGATCTACACGTAAGGAGTCGTCGGCAGCGTC |
| | | (SEQ ID NO: 56) |
| TCL-NIP-A506 | Index Primer A506 | AATGATACGGCGACCACCGAGATCTACACACTGCATATCGTCGGCAGCGTC |
| | | (SEQ ID NO: 57) |
| TCL-NIP-A507 | Index Primer A507 | AATGATACGGCGACCACCGAGATCTACACAAGGAGTATCGTCGGCAGCGTC |
| | | (SEQ ID NO: 58) |
| TCL-NIP-A508 | Index Primer A508 | AATGATACGGCGACCACCGAGATCTACACCTAAGCCTTCGTCGGCAGCGTC |
| | | (SEQ ID NO: 59) |
| TCL-B-For | Biotin-T7-Adapter-B | ATAATACGACTCACTATAGGGGGTCTCGTGGGCTCGGAGATGTGTATAAGAGACAG |
| | | (SEQ ID NO: 60) |
| TCL-B-Rev | TA-Adapter-B-Reverse | TACTGTCTCTTATACACATCTCCGAGCCCACGAGACCCCCTATAGTGAGTCGTATTAT |
| | | (SEQ ID NO: 61) |
| | Index Primer B | CAAGCAGAAGACGGCATACGAGAT[i7]GTCTCGTGGGCTCGG |
| | | (SEQ ID NO: 62) |
| TCL-NIP-B701 | Index Primer B701 | CAAGCAGAAGACGGCATACGAGATTAAGGCGAGTCTCGTGGGCTCGG |
| | | (SEQ ID NO: 63) |
| TCL-NIP-B702 | Index Primer B702 | CAAGCAGAAGACGGCATACGAGATCGTACTAGGTCTCGTGGGCTCGG |
| | | (SEQ ID NO: 64) |
| TCL-NIP-B703 | Index Primer B703 | CAAGCAGAAGACGGCATACGAGATAGGCAGAAGTCTCGTGGGCTCGG |
| | | (SEQ ID NO: 65) |
| TCL-NIP-B704 | Index Primer B704 | CAAGCAGAAGACGGCATACGAGATTCCTGAGCGTCTCGTGGGCTCGG |
| | | (SEQ ID NO: 66) |
| TCL-NIP-B705 | Index Primer B705 | CAAGCAGAAGACGGCATACGAGATGGACTCCTGTCTCGTGGGCTCGG |
| | | (SEQ ID NO: 67) |
| TCL-NIP-B706 | Index Primer B706 | CAAGCAGAAGACGGCATACGAGATTAGGCATGGTCTCGTGGGCTCGG |
| | | (SEQ ID NO: 68) |
| TCL-NIP-B707 | Index Primer B707 | CAAGCAGAAGACGGCATACGAGATCTCTCTACGTCTCGTGGGCTCGG |
| | | (SEQ ID NO: 69) |
| TCL-NIP-B708 | Index Primer B708 | CAAGCAGAAGACGGCATACGAGATCAGAGAGGGTCTCGTGGGCTCGG |
| | | (SEQ ID NO: 70) |

### Example 2

### Variation of the Targeted Chromatin Ligation Protocol without Column Purification

In our original iteration of TCL, described in Example 1 (schematic shown in FIG. 1A), a Targeted Chromatin Ligation reaction was described for use with 200-2000 cells, performed in a tube. That protocol incorporated a column purification step, which was used to clean up DNA for subsequent PCR amplification. However, that purification step reduces throughput, limits automation, and reduces sensitivity.

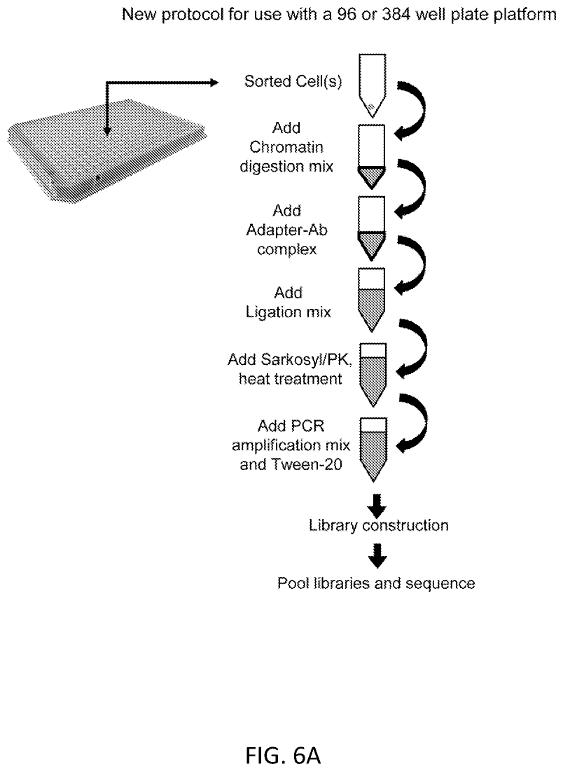

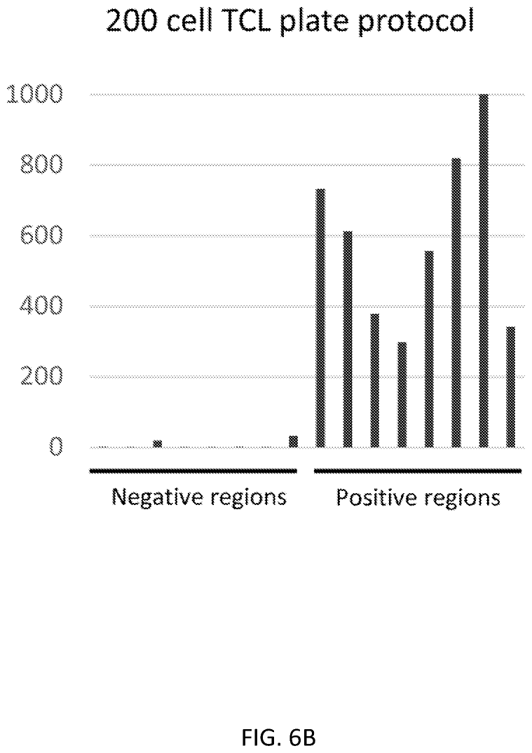

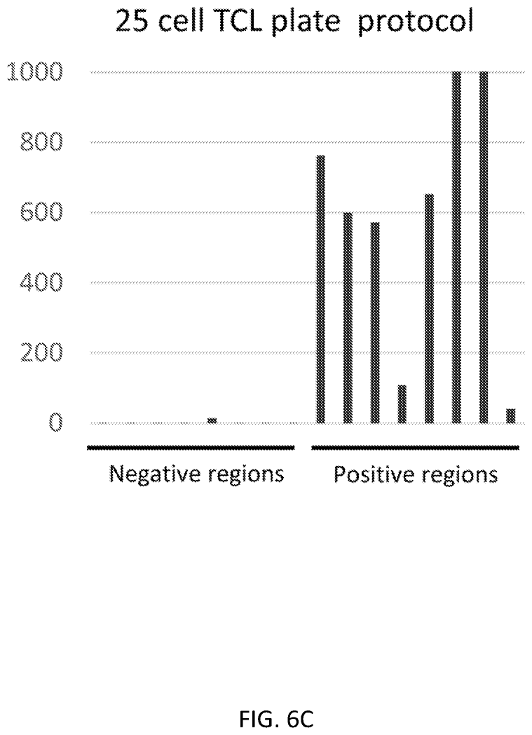

To enable maximum sensitivity of TCL, and to allow for automation and higher throughput, we successfully optimized TCL to remove the column cleanup step and to reduce total volumes needed to perform the TCL reaction (FIGS. 6A-6C). TCL reactions can now be performed in 1/10 the volume with all steps through PCR amplification being performed without changing tubes/wells. We have shown that the new protocol works effectively with as few as 25 cells, versus the 200 cells we reported previously, and believe these methods can be adapted for a single cell reaction.

While the volumes involved are reduced, the new protocol is essentially the same as the previous version, but instead of column purifying DNA after ligation, we now dilute the sample with a new PCR amplification mix (Phusion Blood II polymerase instead of Q5 enzyme mix) and a Tween-20 solution.

For example:

Original protocol: 1) digest cells in 10 ml, incubate. 2) Add 10 ml stop solution. 3) Add 3-5 ml antibody and adapter, incubate. 4) Add 180 ml ligation mix, incubate. 5) Add 20 ml sarkosyl and PK, incubate. 6) Column purify DNA. 7) Perform amplification with Q5 enzyme (volumes arbitrary after step 6). 7) Purify amplified material and make library for sequencing.

New protocol: 1) digest cells in 1 ml, incubate. 2) Add 1 ml stop solution. 3) Add 0.5 ml antibody and adapter, incubate. 4) Add 5 ml ligation mix, incubate. 5) Add 1 ml containing sarkosyl and PK, incubate. 6) Add 38.5 ml amplification mix (15 ml of 15% Tween-20+23 ml Phusion Blood II polymerase reaction mix). 7) Purify amplified material and make library for sequencing.

Plate Based Protocol (MCF7 Cells):

Chromatin fragmentation was performed by adding 10 μl of digestion mix (150 μl CDB+8 μl PI+4 μl EM) to a 2000 cell pellet (spun down at ˜1000 G for 10 minutes) in 1.7 ml tubes (Axygen MCT-175-C). Cells were resuspended by pipetting ˜10×. 1 μl containing 200 cells of material, and 1 μl of a 1:8 dilution, containing 25 cells of material, were then aliquoted to individual wells of a 96 well plate and incubated for 30 minutes at 37° C. using a 96 well plate compatible PCR machine. Digestion was stopped by addition of an equal volume of TDB. 0.5 μl of antibody-adapter complex was added to each TCL sample, mixed by pipetting ˜10×, and then samples were placed at 4° C. overnight without mixing. For MCF7 TCLs, the recommended amounts of antibody bound by adapter are: ˜20 ng anti-H3K27me3.

The next day, the 96 well plate were placed on the work bench and allowed to reach room temperature (˜15 minutes). 5 μl of ligation mix (1× ligation buffer+1 unit ligase/50 μl buffer) were added to each well and mixed by pipetting 2×, then samples were incubated for 10 minutes at room temperature. 1 μl of solution (0,7 μl 10% Sarkosyl+0.3 μl proteinase K (10 mg/ml)) was added to each well. Plate was incubated for 40 minutes at 65° C., then 85° C. for 30 minutes to digest protein and inactivate enzymes. To each well, 15 μl of 15% Tween-20 and 23 μl of Phusion Blood II direct PCR mix was added.

Plate based TCL reactions were performed with a single adapter and ˜35-40 cycles of amplification were used. After amplification, samples were purified with ZYMO columns (30 μl EB) then quantified with a Qubit 3.0 and HS dsDNA assay kit. Amplifications typically yielded ˜50-250 ng of DNA for 25-200 cell TCLs.

Although preferred embodiments of the subject invention have been described in some detail, it is understood that obvious variations can be made without departing from the spirit and the scope of the invention as defined herein.

1. A method of performing targeted chromatin ligation, the method comprising:
providing a sample comprising chromatin;
digesting the chromatin with one or more restriction enzymes, wherein cleavage of the chromatin occurs at positions that are not protected from the restriction enzymes by bound proteins;
contacting the chromatin fragments with one or more antibody-adapter complexes, wherein each antibody-adapter complex comprises an antibody that specifically binds to a DNA-binding protein of interest complexed with an oligonucleotide adapter;
ligating the oligonucleotide adapter of each antibody-adapter complex to its antibody-bound chromatin fragment;
removing bound proteins from the chromatin fragments;
amplifying ligated DNA from chromatin fragments having oligonucleotide adapters ligated at an end, wherein amplification is performed with at least one pair of primers that hybridize to the oligonucleotide adapters; and
sequencing the amplified chromatin fragments.

2. The method of claim 1, wherein the one or more restriction enzymes comprise at least one 4-base cutter and at least one 6-base cutter.

3. The method of claim 2, wherein the one or more restriction enzymes comprise three 4-base cutters and one 6-base cutter.

4. The method of claim 3, wherein three 4-base cutters comprise MseI, BfaI, and Csp6I, and the one 6-base cutter comprises Ndel.

5. (canceled)

6. The method of claim 1, wherein the one or more restriction enzymes produce chromatin fragments having identical overhangs, wherein the overhangs are dinucleotide, trinucleotide, or tetranucleotide overhangs.

7. (canceled)

8. The method of claim 6, wherein the oligonucleotide adapter comprises an overhang sequence that is complementary to the identical overhangs of the chromatin fragments.

9. The method of claim 1, wherein the DNA-binding protein of interest is a histone, transcription factor, or DNA modifying enzyme.

10. The method of claim 1, wherein said at least one antibody-adapter complex comprises an antibody selected from the group consisting of an anti-H3K4me3 antibody, an anti-H3K27me3 antibody, an anti-H3K36me3 antibody, and an anti-H3K27ac antibody.

11. The method of claim 1, wherein said removing bound proteins from the chromatin fragments comprises treating the chromatin fragments with a protease.

12. The method of claim 11, wherein the protease is proteinase K or trypsin.

13. The method of claim 1, further comprising mapping sites of chromatin cleavage and locations of fragment sequences in the chromatin.

14. The method of claim 1, further comprising producing a genome-wide profile of the DNA-binding protein of interest.

15-16. (canceled)

17. The method of claim 1, wherein the cell is a plant cell, an animal cell, a fungus cell, or a protist cell.

18. (canceled)

19. The method of claim 1, further comprising lysing the plasma membrane of the eukaryotic cell prior to said digesting the chromatin with one or more restriction enzymes.

20. (canceled)

21. The method of claim 1, wherein the oligonucleotide adapter is a paired-end sequencing adapter or a mate-pair sequencing adapter.

22. The method of claim 1, further comprising generating a paired-end or mate-pair sequencing library.

23. The method of claim 1, wherein at least some chromatin fragments are ligated to oligonucleotide adapters at both DNA ends.

24-25. (canceled)

26. The method of claim 1, wherein the oligonucleotide adapter comprises a first member of a binding pair and the antibody comprises a second member of a binding pair such that noncovalent binding between the first and second members of the binding pair joins the oligonucleotide adapter to the antibody in the antibody-adapter complex.

27-31. (canceled)

32. A method of identifying an agent that modifies chromatin structure, the method comprising:
treating a test sample comprising chromatin with the agent;
providing a control sample comprising chromatin untreated with the agent;
performing targeted chromatin ligation according to the method of claim 1 on the test sample and the control sample; and
comparing the chromatin fragments from the test sample to the chromatin fragments from the control sample, wherein differences in the size or sequence of at least one chromatin fragment or a position of at least one cleavage site in the chromatin indicate that the agent has modified the structure of the chromatin.

33. The method of claim 32, the method further comprising detecting differences in DNA histone modification or transcription factor binding in the test sample and the control sample.


SEQUENCE LISTING (62 sequences)

SEQ ID NO: 1 - DNA, 20 bp, Artificial Sequence
  ORC4-1 forward primer
  source (1)..(20): sequence is synthesized
cagctctcctcctcctcctt

SEQ ID NO: 2 - DNA, 20 bp, Artificial Sequence
  ORC4-1 reverse primer
  source (1)..(20): sequence is synthesized
agtcagagccagaggaaggc

SEQ ID NO: 3 - DNA, 20 bp, Artificial Sequence
  ORC4-2 forward primer
  source (1)..(20): sequence is synthesized
tgttacgtggtgccatgact

SEQ ID NO: 4 - DNA, 20 bp, Artificial Sequence
  ORC4-2 reverse primer
  source (1)..(20): sequence is synthesized
aaggccaaggagtttgaaga

SEQ ID NO: 5 - DNA, 20 bp, Artificial Sequence
  SKAP2-1 forward primer
  source (1)..(20): sequence is synthesized
aagaaggccttttcccttca

SEQ ID NO: 6 - DNA, 20 bp, Artificial Sequence
  SKAP2-1 reverse primer
  source (1)..(20): sequence is synthesized
ttagcattggcatctgcaag

SEQ ID NO: 7 - DNA, 20 bp, Artificial Sequence
  SKAP2-2 forward primer
  source (1)..(20): sequence is synthesized
cactaacctccctccccttc

SEQ ID NO: 8 - DNA, 20 bp, Artificial Sequence
  SKAP2-2 reverse primer
  source (1)..(20): sequence is synthesized
tggggtagctgtcaccaaat

SEQ ID NO: 9 - DNA, 20 bp, Artificial Sequence
  ELF5-1 forward primer
  source (1)..(20): sequence is synthesized
agctaagggatccagaggga

SEQ ID NO: 10 - DNA, 20 bp, Artificial Sequence
  ELF5-1 reverse primer
  source (1)..(20): sequence is synthesized
gtctgctcgtccatctccag

SEQ ID NO: 11 - DNA, 20 bp, Artificial Sequence
  ELF5-2 forward primer
  source (1)..(20): sequence is synthesized
gtgcgtttggtgaggaattt

SEQ ID NO: 12 - DNA, 20 bp, Artificial Sequence
  ELF5-2 reverse primer
  source (1)..(20): sequence is synthesized
tgggcctataatgctcttcc

SEQ ID NO: 13 - DNA, 20 bp, Artificial Sequence
  RPL19-1 forward primer
  source (1)..(20): sequence is synthesized
gtgctggcatctatgctgaa

SEQ ID NO: 14 - DNA, 20 bp, Artificial Sequence
  RPL19-1 reverse primer
  source (1)..(20): sequence is synthesized
tttgcacacaggcagaaaac

SEQ ID NO: 15 - DNA, 20 bp, Artificial Sequence
  RPL19-2 forward primer
  source (1)..(20): sequence is synthesized
gatgaaagaaccggacagga

SEQ ID NO: 16 - DNA, 20 bp, Artificial Sequence
  RPL19-2 reverse primer
  source (1)..(20): sequence is synthesized
caagaccgacagtcccttgt

SEQ ID NO: 17 - DNA, 20 bp, Artificial Sequence
  ATXN3-1 forward primer
  source (1)..(20): sequence is synthesized
ccacgtccagctactctggt

SEQ ID NO: 18 - DNA, 20 bp, Artificial Sequence
  ATXN3-1 reverse primer
  source (1)..(20): sequence is synthesized
ggcccaagcatttcctttat

SEQ ID NO: 19 - DNA, 20 bp, Artificial Sequence
  ATXN3-2 forward primer
  source (1)..(20): sequence is synthesized
accaggagggcaatatacca

SEQ ID NO: 20 - DNA, 21 bp, Artificial Sequence
  ATXN3-2 reverse primer
  source (1)..(21): sequence is synthesized
tttcttgctgggtcaatagga

SEQ ID NO: 21 - DNA, 20 bp, Artificial Sequence
  USP25-1 forward primer
  source (1)..(20): sequence is synthesized
cgtgtgggaagcagagtgta

SEQ ID NO: 22 - DNA, 20 bp, Artificial Sequence
  USP25-1 reverse primer
  source (1)..(20): sequence is synthesized
tcggttcagaaaggatgaca

SEQ ID NO: 23 - DNA, 20 bp, Artificial Sequence
  USP25-2 forward primer
  source (1)..(20): sequence is synthesized
ctcctcatggcaggttgttt

SEQ ID NO: 24 - DNA, 20 bp, Artificial Sequence
  USP25-2 reverse primer
  source (1)..(20): sequence is synthesized
tcattcgattcgtccctctc

SEQ ID NO: 25 - DNA, 20 bp, Artificial Sequence
  ZEB2-1 forward primer
  source (1)..(20): sequence is synthesized
gcctccttctcctttgcttt

SEQ ID NO: 26 - DNA, 20 bp, Artificial Sequence
  ZEB2-1 reverse primer
  source (1)..(20): sequence is synthesized
ccaggaacctagaatggcac

SEQ ID NO: 27 - DNA, 20 bp, Artificial Sequence
  ZEB2-2 forward primer
  source (1)..(20): sequence is synthesized
gccaggatccctctatttcc

SEQ ID NO: 28 - DNA, 20 bp, Artificial Sequence
  ZEB2-2 reverse primer
  source (1)..(20): sequence is synthesized
caaggctcccagaagtgttc

SEQ ID NO: 29 - DNA, 20 bp, Artificial Sequence
  HOXA1-1 forward primer
  source (1)..(20): sequence is synthesized
cgcgtcaggtacttgttgaa

SEQ ID NO: 30 - DNA, 20 bp, Artificial Sequence
  HOXA1-1 reverse primer
  source (1)..(20): sequence is synthesized
acatttccgtctcatggctt

SEQ ID NO: 31 - DNA, 20 bp, Artificial Sequence
  HOXA1-2 forward primer
  source (1)..(20): sequence is synthesized
cgcacgactggaaagttgta

SEQ ID NO: 32 - DNA, 20 bp, Artificial Sequence
  HOXA1-2 reverse primer
  source (1)..(20): sequence is synthesized
cccatggaggaagtgagaaa

SEQ ID NO: 33 - DNA, 20 bp, Artificial Sequence
  EHF-1 forward primer
  source (1)..(20): sequence is synthesized
attcagccatccagacaacc

SEQ ID NO: 34 - DNA, 20 bp, Artificial Sequence
  EHF-1 reverse primer
  source (1)..(20): sequence is synthesized
atcctcttctctccggcaac

SEQ ID NO: 35 - DNA, 20 bp, Artificial Sequence
  EHF-2 forward primer
  source (1)..(20): sequence is synthesized
tagcgatctggaaacaggct

SEQ ID NO: 36 - DNA, 20 bp, Artificial Sequence
  EHF-2 reverse primer
  source (1)..(20): sequence is synthesized
gggcctgtttgggtttattt

SEQ ID NO: 37 - DNA, 20 bp, Artificial Sequence
  SLC24A4-1 forward primer
  source (1)..(20): sequence is synthesized
tgatgatgtggtttgcccta

SEQ ID NO: 38 - DNA, 20 bp, Artificial Sequence
  SLC24A4-1 reverse primer
  source (1)..(20): sequence is synthesized
ccatgctttcaccaaatcct

SEQ ID NO: 39 - DNA, 20 bp, Artificial Sequence
  SLC24A4-2 forward primer
  source (1)..(20): sequence is synthesized
agcgggttctgatgtcaatc

SEQ ID NO: 40 - DNA, 20 bp, Artificial Sequence
  SLC24A4-2 reverse primer
  source (1)..(20): sequence is synthesized
tcacgaatggacacaccagt

SEQ ID NO: 41 - DNA, 20 bp, Artificial Sequence
  SKAP1-1 forward primer
  source (1)..(20): sequence is synthesized
tcttttccttgcaccttgct

SEQ ID NO: 42 - DNA, 20 bp, Artificial Sequence
  SKAP1-1 reverse primer
  source (1)..(20): sequence is synthesized
gggcatgttgaccagagact

SEQ ID NO: 43 - DNA, 20 bp, Artificial Sequence
  SKAP1-2 forward primer
  source (1)..(20): sequence is synthesized
cactaacctccctccccttc

SEQ ID NO: 44 - DNA, 20 bp, Artificial Sequence
  SKAP1-2 reverse primer
  source (1)..(20): sequence is synthesized
tggggtagctgtcaccaaat

SEQ ID NO: 45 - DNA, 20 bp, Artificial Sequence
  GRIK1-1 forward primer
  source (1)..(20): sequence is synthesized
cttgctgaaatgtggcagaa

SEQ ID NO: 46 - DNA, 20 bp, Artificial Sequence
  GRIK1-1 reverse primer
  source (1)..(20): sequence is synthesized
cttgctgaaatgtggcagaa

SEQ ID NO: 47 - DNA, 20 bp, Artificial Sequence
  GRIK1-2 forward primer
  source (1)..(20): sequence is synthesized
aagtcggaggagggagaaag

SEQ ID NO: 48 - DNA, 20 bp, Artificial Sequence
  GRIK1-2 reverse primer
  source (1)..(20): sequence is synthesized
atccaccatcggatctcttg

SEQ ID NO: 49 - DNA, 55 bp, Artificial Sequence
  Biotin-T7-Adapter-A
  source (1)..(55): sequence is synthesized
ataatacgactcactataggggtcgtcggcagcgtcagatgtgtataagagacag

SEQ ID NO: 50 - DNA, 57 bp, Artificial Sequence
  TA-Adapter-A-Reverse
  source (1)..(57): sequence is synthesized
tactgtctcttatacacatctgacgctgccgacgacccctatagtgagtcgtattat

SEQ ID NO: 51 - DNA, 43 bp, Artificial Sequence
  Index Primer A
  source (1)..(43): sequence is synthesized
aatgatacggcgaccaccgagatctacactcgtcggcagcgtc

SEQ ID NO: 52 - DNA, 51 bp, Artificial Sequence
  Index Primer A501
  source (1)..(51): sequence is synthesized
aatgatacggcgaccaccgagatctacactagatcgctcgtcggcagcgtc

SEQ ID NO: 53 - DNA, 51 bp, Artificial Sequence
  Index Primer A502
  source (1)..(51): sequence is synthesized
aatgatacggcgaccaccgagatctacacctctctattcgtcggcagcgtc

SEQ ID NO: 54 - DNA, 51 bp, Artificial Sequence
  Index Primer A503
  source (1)..(51): sequence is synthesized
aatgatacggcgaccaccgagatctacactatcctcttcgtcggcagcgtc

SEQ ID NO: 55 - DNA, 51 bp, Artificial Sequence
  Index Primer A504
  source (1)..(51): sequence is synthesized
aatgatacggcgaccaccgagatctacacagagtagatcgtcggcagcgtc

SEQ ID NO: 56 - DNA, 51 bp, Artificial Sequence
  Index Primer A505
  source (1)..(51): sequence is synthesized
aatgatacggcgaccaccgagatctacacgtaaggagtcgtcggcagcgtc

SEQ ID NO: 57 - DNA, 51 bp, Artificial Sequence
  Index Primer A506
  source (1)..(51): sequence is synthesized
aatgatacggcgaccaccgagatctacacactgcatatcgtcggcagcgtc

SEQ ID NO: 58 - DNA, 51 bp, Artificial Sequence
  Index Primer A507
  source (1)..(51): sequence is synthesized
aatgatacggcgaccaccgagatctacacaaggagtatcgtcggcagcgtc

SEQ ID NO: 59 - DNA, 51 bp, Artificial Sequence
  Index Primer A508
  source (1)..(51): sequence is synthesized
aatgatacggcgaccaccgagatctacacctaagccttcgtcggcagcgtc

SEQ ID NO: 60 - DNA, 56 bp, Artificial Sequence
  Biotin-T7-Adapter-B
  source (1)..(56): sequence is synthesized
ataatacgactcactatagggggtctcgtgggctcggagatgtgtataagagacag

SEQ ID NO: 61 - DNA, 58 bp, Artificial Sequence
  TA-Adapter-B-Reverse
  source (1)..(58): sequence is synthesized
tactgtctcttatacacatctccgagcccacgagaccccctatagtgagtcgtattat

SEQ ID NO: 62 - DNA, 39 bp, Artificial Sequence
  Index Primer B
  source (1)..(39): sequence is synthesized
caagcagaagacggcatacgagatgtctcgtgggctcgg